(19) United States
(12) Patent Application Publication
(10) Pub. No.: US 2015/0317458 A1
(43) Pub. Date: Nov. 5, 2015

(54) IN SILICO PREDICTION OF ENHANCED NUTRIENT CONTENT IN PLANTS BY METABOLIC MODELLING

(71) Applicant: BASF PLANT SCIENCE COMPANY GMBH, Ludwigshafen (DE)

(72) Inventors: Katrin Lotz, Halle (DE); Michael Leps, Blankenburg (DE); Rainer Lemke, Quedlinburg (DE); Bjoern Junker, Quedlinburg (DE); Falk Schreiber, Quedlingburg (DE)

(73) Assignee: BASF PLANT SCIENCE COMPANY GMBH, Ludwigshafen (DE)

(21) Appl. No.: 14/650,059

(22) PCT Filed: Dec. 5, 2013
(86) PCT No.: PCT/IB2013/060656

§ 371 Date: Jun. 5, 2015

Related U.S. Application Data
(60) Provisional application No. 61/733,924, filed on Dec. 6, 2012.

(30) Foreign Application Priority Data
Dec. 6, 2012 (EP) 12195896.1

(51) Int. Cl. G06F 19/00; C12N 15/82

(52) U.S. Cl. CPC G06F 19/701; C12N 15/8216

(57) ABSTRACT

The present invention relates to a method for identifying at least one metabolic conversion step, the modulation of which increases the amount of a metabolite of interest in a plant cell, plant or plant part, said method comprising establishing a stoichiometric network model for the metabolism of the plant cell, plant or plant part including the synthesis pathway for the metabolite of interest, identifying at least one candidate metabolic conversion step by applying at least one algorithm of Growth-coupled Design, and validating the at least one candidate metabolic conversion step by a constraint-based modeling approach in the stoichiometric network model, wherein an increase in the metabolite of interest occurring in said constraint-based modeling approach is indicative for a metabolic conversion step, the modulation of which increases the amount of the metabolite of interest in the plant cell, plant or plant part. The present invention further relates to a method for generating a plant cell, plant or plant part which produces an increased amount of a metabolite of interest when compared to a control, said method comprising identifying a metabolic conversion step, the modulation of which increases a metabolite of interest in a plant cell, plant or plant part, by the method for identifying a metabolic conversion step and modulating the said metabolic conversion step such that the amount of the metabolite of interest is increased in vivo in a plant cell, plant or plant part.

The present invention relates to a method for identifying at least one metabolic conversion step, the modulation of which increases the amount of a metabolite of interest in a plant cell, plant or plant part, said method comprising: establishing a stoichiometric network model for the metabolism of the plant cell, plant or plant part including the synthesis pathway for the metabolite of interest, identifying at least one candidate metabolic conversion step by applying at least one algorithm of Growth-coupled Design, and validating the at least one candidate metabolic conversion step by a constraint-based modeling approach in the stoichiometric network model, wherein an increase in the metabolite of interest occurring in said constraint-based modeling approach is indicative for a metabolic conversion step, the modulation of which increases the amount of the metabolite of interest in the plant cell, plant or plant part. The present invention further relates to a method for generating a plant cell, plant or plant part which produces an increased amount of a metabolite of interest when compared to a control, said method comprising: identifying a metabolic conversion step, the modulation of which increases a metabolite of interest in a plant cell, plant or plant part, by the method for identifying a metabolic conversion step and modulating the said metabolic conversion step such that the amount of the metabolite of interest is increased in vivo in a plant cell, plant or plant part.

Higher plants are the major source of food and feed, cereal seeds being the basis of nutrition for a large percentage of the human population. However, the composition of cereal seeds, e.g., rice seeds, is not optimal for human and livestock nutrition, since they often comprise suboptimal amounts of compounds essential for animals and man like, e.g, vitamins, amino acids, or unsaturated fatty acids. Means and methods of obtaining cereal plants producing seeds with an optimized content in certain metabolic compounds are thus needed.

The metabolism of an organism of interest can in principle be modelled in silico by establishing a metabolic network model for said organism, e.g. a stoichiometric network model (e.g. Grafahrend-Belau E., Schreiber, F., Koschützki D., Junker B. H. (2009) Plant Physiology. 149(1), 585-598). This, however, requires profound knowledge on the metabolism of said organism. On the basis of such a model, the flow of metabolites through the network can be calculated in a constraint-based modelling approach like flux-balance analysis for steady state analysis (e.g. Orth J. D., Thiele I., Palsson B. O. (2010) Nature Biotechnology. 28(3), 245-248) or like MOMA (Minimization Of Metabolic Adjustment; Segre D., Vitkup D., Church G. M. (2002) PNAS. 99(23), 15112-15117) or ROOM (Regulatory On/Off Minimization; Shlomi T., Berkman O., Ruppin E. (2005) PNAS. 102(21), 7695-7700) for simulating the distortions within the network caused by the loss of a metabolic conversion step, e.g., by a knockout.

There are different public resources available for collection of biochemical data for plant metabolism needed for the reconstruction of different types of metabolic models. The biochemistry of plant metabolism, especially the primary metabolism, has been studied for many years and can be reviewed in principle in many biochemistry text books. In addition, there are several publicly available databases and online resources existing that contain biochemical data about metabolic reactions and it's occurrence and localization in plants (see Table 1).

TABLE 1
Different data sources for biochemical information
about plant metabolism. The resources are characterized
by reaction properties needed for the reconstruction
of plant-specific metabolic models.

| Data source | KEGG | BRENDA | MetaCrop | PlantCyc |
|---|---|---|---|---|
| Stoichiometry | ✓ | ✓ | ✓ | ✓ |
| Directionality | | | ✓ | |
| Localization | | | ✓ | |
| Ontology | ✓ | ✓ | ✓ | ✓ |
| Kinetics | | ✓ | ✓ | |
| References | | ✓ | ✓ | |

The following databases contain almost all necessary biochemical information for plant-specific metabolic models: MetaCrop (Grafahrend-Belau et al., Metacrop: a detailed database for crop plant metabolism. Nucleic Acids Research, 36 (S1):D954-D958, 2008), PlantCyc (Plant Metabolic Network (PNM), 2012, Internet only) and KEGG (Kanehisa and Goto, Kegg: Kyoto encyclopedia of genes and genomes. Nucleic Acids Research, 28(1):27-30, 2000.). All of them support the graphical entrance via organism or pathway specific metabolic network maps whereas the first two contain only plant specific data. KEGG and PlantCyc are highly recommend for getting a system-wide introduction into metabolism: what pathways are present in plants and which reactions are involved. In comparison, MetaCrop is a hand-curated database which contains additional information about reaction directionality and reaction's compartmental localization and their respective references. But MetaCrop does not contain all known metabolic pathways occurring in plants and therefore also BRENDA (Scheer et al., Brenda, the enzyme information system in 2011. Nucleic Acids Research, 39 (suppl 1):D670-D676, 2010.) is very useful by providing organism-specific references for all enzymatic reactions in almost all plant species, if available.

Based on the available biochemical information for the plant of interest the metabolic model can be reconstructed in order to analyse the network structure, calculate feasible flux distributions or explore dynamic properties of the metabolic system.

Based on the models detailed above, algorithms have been devised to solve the bilevel optimization problem of optimizing the production of a metabolite of interest while maintaining a suitable growth rate for the relatively simple metabolic networks of bacteria. These algorithms are able to propose knockout strategies for implementing said optimization (see e.g. Burgard A. P., Pharkya P., Maranas C. D. (2003) Biotechnology and Bioengineering. 84(6):647-657; Tepper N., Shlomi T. (2010) Bioinformatics. 26(4):536-543). However, for the complex metabolism of plants, prediction of knockouts suitable for changing the concentration of a metabolite of interest is a challenge still today. Thus, there is a need for the reliable prediction of metabolic effects. The technical problem underlying the present invention could, thus, be seen as the provision of means and methods for making predictions of relevant metabolic effects and for, thereby, allowing to identify metabolic conversion steps in a metabolism for the production of a metabolite of interest. The technical problem is solved by the embodiments characterized in the claims and herein below.

Accordingly, the present invention relates to a method for identifying at least one metabolic conversion step, the modulation of which increases the amount of a metabolite of interest in a plant cell, plant or plant part, said method comprising: (a) establishing a stoichiometric network model for the metabolism of the plant cell, plant or plant part including the synthesis pathway for the metabolite of interest; (b) identifying at least one candidate metabolic enzymatic conversion step by applying at least one algorithm of Growth-coupled Design; and (c) validating the at least one candidate metabolic conversion step by a constraint-based modeling approach in the stoichiometric network model, wherein an increase in the metabolite of interest occurring in said constraint-based modeling approach is indicative for a metabolic conversion step, the modulation of which increases the amount of the metabolite of interest in the plant cell, plant or plant part.

The method for identifying at least one metabolic conversion step of the present invention, preferably, is an in-silico method. Thus, preferably, most or all of the steps of said method are performed in a computer-assisted mode. Moreover, said method may comprise further steps in addition to the ones explicitly mentioned. Specifically, step a) may, preferably, comprise the further step of generating and/or collecting data required to establish a stoichiometric network model for the metabolism in question or step c) may, preferably, contain the further steps of validating the metabolic conversion step by constructing and analyzing a plant comprising a mutation of the gene encoding the enzyme catalyzing said metabolic conversion step as described herein below.

The term “metabolic conversion step”, as used herein, relates to any chemical or physical modification of a compound comprised by a plant, plant part, plant organ, or plant cell. Preferably, the metabolic conversion step is a chemical conversion of a compound into a chemically different compound. More preferably, the metabolic conversion step is an enzymatically catalyzed chemical reaction. Most preferably, the metabolic conversion step is a chemical reaction catalyzed by a polypeptide having enzymatic properties expressed by the plant cell, i.e. an enzymatic conversion. It is to be understood that the term may refer to any conversion in the metabolism of a plant, including e.g., anabolism, catabolism, and secondary metabolism. It is also to be understood that the term may also refer to the translocation or transport of a compound within the plant of the present invention. Preferably, included by the term metabolic conversion step are, thus, the transport of a compound in the xylem or phloem of a plant, or the transport from one cell compartment into another, preferably, over one or more cellular membranes.

As used herein, the term “plant” relates to a whole plant, a plant part, a plant organ, a plant tissue, or a plant cell. Thus the term includes, preferably, seeds, shoots, stems, leaves, roots (including tubers), and flowers. Plants that are particularly useful in the methods of the invention include all plants which belong to the superfamily Viridiplantae, preferably Tracheophyta, more preferably Spermatophytina, most preferably monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

The term “modulation”, as used herein, relates to a change of a stoichiometric or kinetic parameter of a metabolic conversion step from the corresponding parameter found under physiological conditions in a plant cell, plant, or plant part. Physiological conditions are those which can be observed without modulation of the step. Preferably, the said change is a statistically significant change. The change may be an increase or a decrease. The modulation of a metabolic conversion step and thus, the deviation of a stoichiometric parameter can, e.g., be achieved by deleting or mutating a gene encoding a subunit of an enzyme complex catalyzing a partial reaction of an enzymatic step, such that the amount or identity of the final product is altered. A deviation of a kinetic parameter can, e.g., be achieved by deleting the gene coding for an enzyme catalyzing the metabolic conversion step in question, such that the reaction velocity is reduced to the reaction velocity of the uncatalyzed conversion, which is, preferably, zero. Preferably, modulation encompasses decreasing or increasing the activity of an enzyme catalyzing said metabolic conversion. More preferably, modulation is abolishing the activity of an enzyme catalyzing said metabolic conversion step. Preferably, modulation is achieved by modulation of gene expression. Thus, preferably, the term “modulation” means in relation to expression or gene expression, a process or state in which the level of gene expression is changed by said process or state in comparison to the control plant, wherein the expression level may be increased or decreased. The original, unmodulated expression may be of any kind of expression of a structural RNA (rRNA, tRNA) or mRNA with subsequent translation. The term “modulating the activity” in relation to expression or gene expression shall mean any change of the expression of the gene, leading to an altered concentration of the corresponding polynucleotides or encoded proteins in the cell.

Modulation of an enzymatic activity can be achieved by a variety of methods well known in the art.

Preferably, the modulation is an activation, i.e., preferably, a modulation increasing the activity of an enzyme catalyzing said metabolic conversion. Activation can, preferably, be achieved by application of an activator for the enzyme. More preferably, activation is mediated by introducing into the plant cell one or more molecules of an enzyme catalyzing said metabolic conversion step. Said enzyme may, preferably, be autologous or, more preferably, heterologous. Said enzyme, may be a wildtype enzyme or a mutated enzyme with an increased activity. Also, the enzyme may be introduced into the plant cell as a polypeptide or, more preferably, as an expressible gene.

The term “expression” or “gene expression” relates to transcription of a specific gene or specific genes or a specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product. The term “increased expression” or “overexpression” as used herein means any form of expression that is additional to the original wild-type expression level. Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene. If polypeptide expression is desired, it is generally desirable to include a polyadenylation region at the 3′-end of a polynucleotide coding region. The polyadenylation region can, preferably, be derived from the natural gene, from a variety of other plant genes, or from T-DNA, and the like. The 3′ end sequence to be added may be derived from, for example, the nopaline synthase or octopine synthase genes, or alternatively from another plant gene, or, less preferably, from any other eukaryotic gene. An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

Also preferably, the modulation is an inactivation or inhibition, i.e., preferably, a modulation decreasing the activity of an enzyme catalyzing said metabolic conversion. Preferably, the inhibition is reversible, more preferably the inhibition is irreversible, i.e. an inactivation. A direct inhibition is achieved by a compound which binds to the enzyme and thereby inhibits its catalytic activity. Compounds which directly inhibit enzymes in this sense are, preferably, compounds which block the interaction of the enzyme with other proteins or with its substrates. Alternatively, but nevertheless preferred, a direct inhibitor of an enzyme may induce an allosteric change in the conformation of the polypeptide constituting the enzyme. The allosteric change may subsequently block the interaction of the enzyme with other proteins or with its substrates and, thus, interfere with the catalytic activity of the enzyme. Compounds which are suitable as direct inhibitors of enzymes encompass small molecule antagonists (e.g., substrate analogues, allosteric inhibitors), antibodies, aptamers, mutants or variants of the enzyme, a dominant-negative subunit of an enzyme complex, and the like.

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

The term “small molecule antagonist” as used herein refers to a chemical compound that specifically interacts and inhibits the enzyme. A small molecule as used herein preferably has a molecular weight of less than 1000 Da, more preferably, less than 800 Da, less than 500 Da, less than 300 Da, or less than 200 Da. Such small molecules are, preferably, capable of diffusing across cell membranes so that they can enter and reach intracellular sites of action. Suitable chemical compounds encompass small organic molecules. Preferably, the small molecule antagonist is a substrate analogon or an allosteric inhibitor.

The term “antibody” as used herein encompasses all types of an antibody which, preferably, specifically binds to an enzyme and inhibits its activity. Preferably, the antibody of the present invention is a monoclonal antibody, a polyclonal antibody, a single chain antibody, a chimeric antibody or any fragment or derivative of such antibodies being still capable of binding to the enzyme and inhibiting its catalytic activity. Such fragments and derivatives comprised by the term antibody as used herein encompass a bispecific antibody, a synthetic antibody, an Fab, F(ab)2 Fv or scFv fragment, or a chemically modified derivative of any of these antibodies. Specific binding as used in the context of the antibody of the present invention means that the antibody does not cross-react with other polypeptides or, preferably, does not inhibit the activity of other polypeptides. Specific binding and/or inhibition can be tested by various well known techniques. Inhibition is preferably tested by an enzymatic assay determining the activity of the enzyme in question in the presence and in the absence of the antibody. Antibodies or fragments thereof, in general, can be obtained by using methods which are described well known to the skilled person. Monoclonal antibodies can be prepared the techniques which comprise the fusion of mouse myeloma cells to spleen cells derived from immunized mammals and, preferably, immunized mice. Monoclonal antibodies which specifically bind to the enzyme can be prepared using the well known hybridoma technique, the human B cell hybridoma technique, and the EBV hybridoma technique. Specifically binding antibodies which affect at least one catalytic activity can be identified by assays known in the art.

The term “aptamer” as used herein relates to oligonucleic acid or peptide molecules that bind to a specific target polypeptide. Oligonucleic acid aptamers are engineered through repeated rounds of selection or the so called systematic evolution of ligands by exponential enrichment (SELEX technology). Peptide aptamers are designed to interfere with protein interactions inside cells. They usually comprise of a variable peptide loop attached at both ends to a protein scaffold. This double structural constraint shall increase the binding affinity of the peptide aptamer into the nanomolar range. Said variable peptide loop length is, preferably, composed of ten to twenty amino acids, and the scaffold may be any protein having improved solubility and compacity properties, such as thioredoxin-A. Peptide aptamer selection can be made using different systems including, e.g., the yeast two-hybrid system. Aptamers which affect at least one biological activity of an enzyme can be identified by functional assays known in the art.

The term “dominant-negative subunit of an enzyme complex”, as used herein, refers to a subunit of an enzyme complex mutated such that it is still able to bind to the enzyme complex, but not catalytically active. Thus, the non-catalytic dominant-negative subunit disclocates a functional subunit from the complex, leading to a decreased, altered, or abolished activity of the complex.

Inhibition of an enzyme according to the present invention is, preferably, achieved by indirect inhibition wherein the number of molecules of said enzyme present in a plant cell is reduced. Preferably, the number of molecules of said enzyme is reduced to zero, i.e. production of enzyme molecules is abolished. Such a reduction of the number of enzyme molecules is, preferably, accomplished by a reduction or prevention of the expression of the gene coding for said enzyme, i.e. by a reduction or prevention of transcription, a destabilization or increased degradation of the transcripts or a reduction or prevention of the translation of the transcripts into enzyme polypeptides. Compounds which are known to interfere with transcription and/or translation of genes as well as stability of transcripts are inhibitory nucleic acids. Such inhibitory nucleic acids, usually, recognize their target transcripts by hybridization of nucleic acid sequences present in both, the target transcript and the inhibitory nucleic acid, being complementary to each other. Accordingly, for a given transcript with a known nucleic acid sequence, such inhibitors can be designed and synthesized without further ado by the skilled artisan. Suitable assays for testing the activity are known in the art. Specifically, the presence or absence of the target transcript can be measured or the presence or absence of the protein encoded thereby, or its activity, can be measured in the presence and absence of the putative inhibitory nucleic acid. A nucleic acid which, indeed, is an inhibitory nucleic acid can be subsequently identified if in the presence of the inhibitory nucleic acid, the target transcript, the polypeptide, or the enzymatic activity encoded thereby can no longer be detected or is detectable at reduced amounts.

Reference herein to “reducing the number of enzyme molecules” or “reduction or substantial elimination” is taken to mean a decrease in endogenous gene expression and polypeptide levels and/or polypeptide activity relative to control plants. The reduction or substantial elimination is, preferably to a statistically significant extent and, more preferably, in increasing order of preference a reduction of at least 10%, 20%, 30%, 40% or 50%, 60%, 70%, 80%, 85%, 90%, or 95%, 96%, 97%, 98%, 99% or more compared to that of control plants.

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants.

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid encoding the protein of interest (target gene), or from any nucleic acid capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, 90%, 95%, 96%, 97%, 98%, 99%, 100% sequence identity to the target gene (either sense or antisense strand). A nucleic acid sequence encoding a (functional) polypeptide is not a requirement for the various methods discussed herein for the reduction or substantial elimination of expression of an endogenous gene.

This reduction or substantial elimination of expression may be achieved using routine tools and techniques. A preferred method for the reduction or substantial elimination of endogenous gene expression is by introducing and expressing in a plant a genetic construct into which the nucleic acid (in this case a stretch of substantially contiguous nucleotides derived from the gene of interest, or from any nucleic acid capable of encoding an orthologue, paralogue or homologue of any one of the protein of interest) is cloned as an inverted repeat (in part or completely), separated by a spacer (non-coding DNA).

Accordingly, the inhibitor of the invention is, preferably, an inhibitory nucleic acid. More preferably, said inhibitory nucleic acid is selected from the group consisting of: an antisense RNA, a ribozyme, a siRNA, a micro RNA, a morpholino or a triple helix forming agent.

The term “antisense RNA” as used herein refers to an RNA which comprises a nucleic acid sequence which is essentially or perfectly complementary to the target transcript. Preferably, an antisense nucleic acid molecule essentially consists of a nucleic acid sequence being complementary to at least 100 contiguous nucleotides, more preferably, at least 200, at least 300, at least 400 or at least 500 contiguous nucleotides of the target transcript. How to generate and use antisense nucleic acid molecules is well known in the art (see, e.g., Weiss, B. (ed.): Antisense Oligodeoxynucleotides and Antisense RNA: Novel Pharmacological and Therapeutic Agents, CRC Press, Boca Raton, Fla., 1997.). The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The term “ribozyme” as used herein refers to catalytic RNA molecules possessing a well defined tertiary structure that allows for catalyzing either the hydrolysis of one of their own phosphodiester bonds (self-cleaving ribozymes), or the hydrolysis of bonds in other RNAs, but they have also been found to catalyze the aminotransferase activity of the ribosome. The ribozymes envisaged in accordance with the present invention are, preferably, those which specifically hydrolyse the target transcripts. In particular, hammerhead ribozymes are preferred in accordance with the present invention. How to generate and use such ribozymes is well known in the art (see, e.g., Hean J, Weinberg M S (2008). “The Hammerhead Ribozyme Revisited: New Biological Insights for the Development of Therapeutic Agents and for Reverse Genomics Applications”. In Morris K L. RNA and the Regulation of Gene Expression: A Hidden Layer of Complexity. Norfolk, England: Caister Academic Press).

The term “siRNA” as used herein refers to small interfering RNAs (siRNAs) which are complementary to target RNAs (encoding a gene of interest) and diminish or abolish gene expression by RNA interference (RNAi). Without being bound by theory, RNAi is generally used to silence expression of a gene of interest by targeting mRNA. Briefly, the process of RNAi in the cell is initiated by double stranded RNAs (dsRNAs) which are cleaved by a ribonuclease, thus producing siRNA duplexes. The siRNA binds to another intracellular enzyme complex which is thereby activated to target whatever mRNA molecules are homologous (or complementary) to the siRNA sequence. The function of the complex is to target the homologous mRNA molecule through base pairing interactions between one of the siRNA strands and the target mRNA. The mRNA is then cleaved approximately 12 nucleotides from the 3′ terminus of the siRNA and degraded. In this manner, specific mRNAs can be targeted and degraded, thereby resulting in a loss of protein expression from the targeted mRNA. A complementary nucleotide sequence as used herein refers to the region on the RNA strand that is complementary to an RNA transcript of a portion of the target gene. The term “dsRNA” refers to RNA having a duplex structure comprising two complementary and anti-parallel nucleic acid strands. Not all nucleotides of a dsRNA necessarily exhibit complete Watson-Crick base pairs; the two RNA strands may be substantially complementary. The RNA strands forming the dsRNA may have the same or a different number of nucleotides, with the maximum number of base pairs being the number of nucleotides in the shortest strand of the dsRNA. Preferably, the dsRNA is no more than 49, more preferably less than 25, and most preferably between 19 and 23, nucleotides in length. dsRNAs of this length are particularly efficient in inhibiting the expression of the target gene using RNAi techniques. dsRNAs are subsequently degraded by a ribonuclease enzyme into short interfering RNAs (siRNAs). The complementary regions of the siRNA allow sufficient hybridization of the siRNA to the target RNA and thus mediate RNAi. In mammalian cells, siRNAs are approximately 21-25 nucleotides in length. The siRNA sequence needs to be of sufficient length to bring the siRNA and target RNA together through complementary base-pairing interactions. The siRNA used with the Tet expression system of the invention may be of varying lengths. The length of the siRNA is preferably greater than or equal to ten nucleotides and of sufficient length to stably interact with the target RNA; specifically 10-30 nucleotides; more specifically any integer between 10 and 30 nucleotides, most preferably 10, 11, 12, 13, 14, 15, 16, 17, 18, 19, 20, 21, 22, 23, 24, 25, 26, 27, 28, 29, and 30. By “sufficient length” is meant an oligonucleotide of greater than or equal to 15 nucleotides that is of a length great enough to provide the intended function under the expected condition. By “stably interact” is meant interaction of the small interfering RNA with target nucleic acid (e.g., by forming hydrogen bonds with complementary nucleotides in the target under physiological conditions). Generally, such complementarity is 100% between the siRNA and the RNA target, but can be less if desired, preferably 91%, 92%, 93%, 94%, 95%, 96%, 97%, 98%, or 99%. For example, 19 bases out of 21 bases may be base-paired. In some instances, where selection between various allelic variants is desired, 100% complementary to the target gene is required in order to effectively discern the target sequence from the other allelic sequence. When selecting between allelic targets, choice of length is also an important factor because it is the other factor involved in the percent complementary and the ability to differentiate between allelic differences. Methods relating to the use of RNAi to silence genes in organisms, including C. elegans, Drosophila, plants, and mammals, are known in the art (see, e.g., WO 0129058; WO 09932619; and Elbashir (2001), Nature 411: 494-498).

The term “microRNA” as used herein refers to a self complementary single-stranded RNA which comprises a sense and an antisense strand linked via a hairpin structure. The micro RNA comprise a strand which is complementary to an RNA targeting sequences comprised by a transcript to be downregulated. micro RNAs are processed into smaller single stranded RNAs and, therefore, presumably also act via the RNAi mechanisms. How to design and to synthesise microRNAs which specifically degrade a transcript of interest is known in the art and described, e.g., in EP 1 504 126 A2 or Dimond (2010), Genetic Engineering & Biotechnology News 30 (6):1.

Another example of an RNA silencing method involves the introduction of nucleic acid sequences or parts thereof (in this case a stretch of substantially contiguous nucleotides derived from the gene of interest, or from any nucleic acid capable of encoding an orthologue, paralogue or homologue of the protein of interest) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

The term “morpholino” refers to a synthetic nucleic acid molecule having a length of 20 to 30 nucleotides, preferably, about 25 nucleotides. Morpholinos bind to complementary sequences of target transcripts by standard nucleic acid base-pairing. They have standard nucleic acid bases which are bound to morpholine rings instead of deoxyribose rings and linked through phosphorodiamidate groups instead of phosphates. The replacement of anionic phosphates with the uncharged phosphorodiamidate groups eliminates ionization in the usual physiological pH range, so morpholinos in organisms or cells are uncharged molecules. The entire backbone of a morpholino is made from these modified subunits. Unlike inhibitory small RNA molecules, morpholinos do not degrade their target RNA molecules. Rather, they sterically block binding to a target sequence within an RNA and simply getting in the way of molecules that might otherwise interact with the RNA (see, e.g., Summerton (1999), Biochimica et Biophysica Acta 1489 (1): 141-58).

The term “triple helix forming agent” as used herein refers to oligonucleotides which are capable of forming a triple helix with DNA and, in particular, which interfere upon forming of the triple-helix with transcription initiation or elongation of a desired target gene such as RAGE in the case of the inhibitor of the present invention. The design and manufacture of triple helix forming agents is well known in the art (see, e.g., Vasquez (2002), Quart Rev Biophys 35: 89-107).

For optimal performance, the gene silencing techniques used for reducing expression in a plant of an endogenous gene require the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Abolishing production of enzyme molecules, i.e. reduction by 100%, is accomplished in a variety of ways. The gene coding for said enzyme can, e.g., be deleted or mutated in a way such that a functional enzyme can no longer be expressed (Knockout-mutation, KO-mutation). Alternatively, said gene may be replaced, e.g. by a non-functional gene, by a mutant copy coding for an inactive variant, or by a gene coding for a selectable marker, e.g., preferably, by homologous recombination. Homologous recombination allows introduction into a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offringa et al. (1990) EMBO J 9(10): 3077-84) but also for crop plants, for example rice (Terada et al. (2002) Nat Biotech 20(10): 1030-4; Iida and Terada (2004) Curr Opin Biotech 15(2): 132-8), and approaches exist that are generally applicable regardless of the target organism (Miller et al, Nature Biotechnol. 25, 778-785, 2007). It is known to the skilled person that such deletion, mutation, or replacement will have to be performed for each copy of the wildtype gene coding for said enzyme available in said plant cell. It is also known to the skilled person that said deletion, mutation, or replacement may, but does not have to, extend to isoenzymes, preferably isoenzymes encoded and/or active in other compartments of the cell. A KO-mutation may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682).

Preferably, a reduction of enzyme molecules is achieved by TILING. The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50).

Alternatively, a screening program may be set up to identify in a plant population natural variants of a gene, which variants encode polypeptides with reduced activity. Such natural variants may also be used for example, to perform homologous recombination.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

The term “significant”, as used in this specification, relates to statistical significance. Whether a data set supports a hypothesis in a statistically significant way can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001.

The term “amount” relates to the quantity of a metabolite or compound of the present invention. Preferably, the amount is determined as the concentration of the metabolite in the cell, as the fraction of biomass or dry mass, or any other method suitable for determining a quantity of a specific substance. An increase in amount is preferably a significant increase, more preferably an increase of the amount is an increase by 2-5%, 5-10%, 10-20%, 20-50%, 50-100%, 10-100%, 100-200%, or 100-500% as compared to a control plant. Most preferably, an increase in amount is an increase by at least 2%, 5%, 10%, 20%, 30%, 40%, 50%, 75%, 100%, 200%, 300%, 400%, or at least 500% as compared to a control plant. The term “biomass” as used herein is intended to refer to the total weight of a plant. Within the definition of biomass, a distinction may be made between the biomass of one or more parts of a plant, which may include any one or more of the following: aboveground parts such as but not limited to shoot biomass, seed biomass, leaf biomass, etc.; aboveground harvestable parts such as but not limited to shoot biomass, seed biomass, leaf biomass, etc.; parts below ground, such as but not limited to root biomass, etc.; harvestable parts below ground, such as but not limited to root biomass, etc.; vegetative biomass such as root biomass, shoot biomass, etc.; reproductive organs; and propagules, such as seed.

As used herein, the term “metabolite of interest” relates to any compound of the primary or secondary metabolism of a plant. Preferably, the metabolite of interest is a compound not synthesized by the body cells of at least one animal species, preferably at least one mammalian species, more preferably at least one livestock species, or, most preferably, man. Preferably, the metabolite of interest is an amino acid, more preferably the metabolite is arginine, cysteine, glycine, glutamine, histidine, proline, serine, tyrosine, phenylalanine, valine, threonine, tryptophan, isoleucine, methionine, leucine, lysine, or histidine, most preferably the L-form of the respective amino acid. Also included as metabolites of interest are, preferably, vitamins, more preferably, Vitamin A (Retinol), Vitamin B1 (Thiamine), Vitamin C (Ascorbic acid), a form of Vitamin D (Calciferol), Vitamin B2 (Riboflavin), Vitamin E (Tocopherol), Vitamin K1 (Phylloquinone), Vitamin B5 (Pantothenic acid), Vitamin B7 (Biotin), B6 (Pyridoxine), Vitamin B3 (Niacin), or Vitamin B9 (Folic acid). Also included as metabolites of interest are, preferably, fatty acid, more preferably, unsaturated fatty acid, most preferably, polyunsaturated fatty acids. Further included as metabolites of interest are, preferably, carbohydrates, more preferably, sugars, starch, and the like.

The term “network model”, as used herein, relates to a representation and simulation of metabolic and physical conversions that determine the physiological and biochemical properties of a plant. Preferably, the network model comprises the metabolic conversions of the synthesis pathway for the metabolite of interest. More preferably, the network model comprises all metabolic conversions having an impact on the amount of the metabolite of interest. The term “having an impact” relates to a metabolic conversion which, when abolished, leads to a deviation from normal of the amount of the metabolite of interest of at least 5%, at least 10%, at least 25%, at least 50%, at least 100%, at least 200%, at least 500%, or at least 1000%. Even more preferably, the network model comprises all metabolic conversions of the complete primary metabolism of the plant, i.e. preferably, the network model comprises all relevant metabolic conversion steps of the anabolic and catabolic pathways of the metabolism of the plant. Most preferably, the network model comprises all known metabolic conversions of a plant. The term “known metabolic conversion”, preferably, includes metabolic conversions known from in silico predictions of enzymes encoded in the genome of said plant.

The term “stoichiometric network model”, as used herein, relates to a network model comprising data related to the stoichiometry of educts and products of the metabolic conversions comprised in said network model. Preferably, the stoichometric network model also comprises data related to the composition of the plant, plant part, plant tissue, or plant cell of interest. It is, thus, understood by the skilled person that a stoichiometric network model, preferably, is specific for a specific plant, plant part, or plant tissue having said composition. More preferably, the stoichiometric network model is a stoichiometric network model of rice, most preferably of rice seeds. In a preferred embodiment, the stoichiometric network model comprises the data of Table 3 below, more preferably, the data of Table 3 and FIG. 1. Abbreviations in Table 3 are explained in Table 4. Preferably, the stoichiometric network model does not comprise kinetic data related to the metabolic conversions. Preferably, the stoichiometric network model is implemented in a data processor, more preferably a computer.

As used herein, the term “algorithm of Growth-coupled Design” relates to an algorithm solving a bilevel optimization, wherein the first optimization is the maximization of the production of the amount of the metabolite of interest, and wherein the second optimization is maintenance of metabolic conversions leading to the production of growth resources. It is understood by the skilled person that the amount of metabolite of interest obtainable, i.e. the first optimization, will depend strongly on the identity of the metabolite of interest. E.g., in case the metabolite is an amino acid, preferably leucine, preferred amounts are at least 0.001 mmol*g dry weight (gDW)⁻¹*h⁻¹, at least 0.002 mmol*gDW⁻¹*h⁻¹, at least 0.003 mmol*gDW⁻¹*h⁻¹, at least 0.004 mmol*gDW⁻¹*h⁻¹, at least 0.005 mmol*gDW⁻¹*h⁻¹, at least 0.01 mmol*gDW⁻¹*h⁻¹, at least 0.02 mmol*gDW⁻¹*h⁻¹, at least 0.05 mmol*gDW⁻¹*h⁻¹, or at least 0.1 mmol*gDW⁻¹*h⁻¹. Preferably, said maintenance of metabolic conversions leading to the production of growth resources, i.e. the second optimization, allows for a growth rate of at least 0.0014/h, at least 0.0019/h, at least 0.0024/h, at least 0.0029/h, at least 0.0034/h, at least 0.0038/h, or at least 0.0043/h. More preferably, said maintenance of metabolic conversions leading to the production of growth resources allows for a growth rate, i.e., preferably, to a biomass production, of at least 0.001 mmol*g dry weight (gDW)⁻¹*h⁻¹, at least 0.002 mmol*gDW⁻¹*h⁻¹, at least 0.003 mmol*gDW⁻¹*h⁻¹, at least 0.004 mmol*gDW⁻¹*h⁻¹, at least 0.005 mmol*gDW⁻¹*h⁻¹, at least 0.01 mmol*gDW⁻¹*h⁻¹, at least 0.02 mmol*gDW⁻¹*h⁻¹, at least 0.05 mmol*gDW⁻¹*h⁻¹, or at least 0.1 mmol*gDW⁻¹*h⁻¹. Preferably, the amount of biomass is calculated based on fixed substrate uptake rates for the metabolic network of the plant cell, plant or plant part and/or the plant-specific nutritional composition in the stoichiometric network model under conditions where at least one metabolic enzymatic conversion step is reduced or enhanced. Preferably, the bilevel optimization is solved by calculating the amount of the metabolite of interest based on the calculated amount of biomass. More preferably, the bilevel optimization is solved by calculating the product of the amount of metabolite of interest and the growth rate obtainable, i.e., preferably, the yield, for a specific modulation or a specific set of modulations. Preferably, the algorithm of Growth-coupled Design is a mathematical algorithm or a genetic algorithm. More preferably, the algorithm of Growth-coupled Design is capable of at least calculating the amount of the metabolite of interest obtained in the stoichiometric network model under conditions where at least one metabolic enzymatic conversion step is reduced and the algorithm of Growth-coupled Design is capable of thereby identifying at least one metabolic enzymatic conversion step the reduction of which yields the maximum amount for the metabolite of interest. Most preferably, the mathematical algorithm is OptKnock or RobustKnock (see Table 2 below) and/or the genetic algorithm is OptGene (see Table 2). In a preferred embodiment, OptKnock and/or RobustKnock are to be used if one to four metabolic enzymatic conversion step(s), the modulation of which increases a metabolite of interest in a plant cell, plant or plant part, shall be identified. In another preferred embodiment, OptGene is to be used if more than four metabolic enzymatic conversion steps, the modulation of which increases a metabolite of interest in a plant cell, plant or plant part, shall be identified. Examples of preferred algorithms, their uses, and relevant publications are shown in table 2. Preferably, the algorithm is implemented in a data processor, more preferably a computer.

As used herein, the term “constraint-based modeling” relates to modeling the metabolism of a plant based on physicochemical constraints and/or reaction stoichiometry constraints arising from the requirement that fluxes consuming and producing metabolites are balanced. Preferably, the term relates to a modeling based on the constraints thermodynamic directionality and/or enzymatic capacity and/or reaction stoichiometry. Preferably, the metabolites considered are low-molecular weight organic compound. More preferably, in addition protons and/or electrons (reducing equivalents) are taken into account in said modeling.

In a preferred embodiment, the present invention relates to the method as described supra, wherein said modulation of a metabolic conversion step encompasses decreasing or increasing the activity of at least one enzyme catalyzing the metabolic conversion step in the plant cell.

In another preferred embodiment, the present invention relates to the method as described supra, wherein said stoichiometric network model for the metabolism of the plant cell, plant or plant part comprises all relevant metabolic conversion steps of the anabolic and catabolic pathways of the metabolism of the plant cell, plant or plant part and wherein each metabolic conversion step is defined by its underlying reaction stoichiometry.

In a further preferred embodiment, the present invention relates to the method as described supra, wherein said at least one algorithm for solving the Growth-coupled Design (i) is capable of at least calculating the amount of the metabolite of interest obtained in the stoichiometric network model under conditions where at least one metabolic enzymatic conversion step is reduced and (ii) is capable of thereby identifying at least one metabolic enzymatic conversion step the reduction of which yields the maximum amount for the metabolite of interest.

In yet another preferred embodiment, the present invention relates to the method as described supra, wherein the amount of the metabolite of interest is calculated based on the calculated amount of biomass.

In an also preferred embodiment, the present invention relates to the method as described supra, wherein said amount of biomass is calculated based on (i) fixed substrate uptake rates for the metabolic network of the plant cell, plant or plant part and/or (ii) the plant-specific nutritional composition in the stoichiometric network model under conditions where at least one metabolic enzymatic conversion step is reduced or enhanced.

In another preferred embodiment, the present invention relates to the method as described supra, wherein said at least one algorithm for solving the Growth-coupled Design is selected from the group consisting of: OptKnock, RobustKnock and OptGene.

In a further preferred embodiment, the present invention relates to the method as described supra, wherein OptKnock and/or RobustKnock are to be used if one to four metabolic enzymatic conversion step(s), the modulation of which increases a metabolite of interest in a plant cell, plant or plant part, shall be identified.

In an also preferred embodiment, the present invention relates to the method as described supra, wherein OptGene is to be used if more than four metabolic enzymatic conversion steps, the modulation of which increases a metabolite of interest in a plant cell, plant or plant part, shall be identified.

In a further preferred embodiment, the present invention relates to the method as described supra, wherein said plant cell, plant or plant part is a rice cell, rice plant, rice plant part, or rice seed.

In yet another preferred embodiment, the present invention relates to the method as described supra, wherein said metabolite of interest is an amino acid, a fatty acid, or a carbohydrate.

In a further preferred embodiment, the present invention relates to the method as described supra, wherein steps (a) to (c) of said method are automated by implementation on a data processing device.

In another preferred embodiment, the present invention relates to the method as described supra, wherein said method further comprises the further step of:

(d) determining whether the metabolic enzymatic conversion step validated in step (c) increases the metabolite of interest in the plant cell, plant or plant part by modulating the said metabolic enzymatic conversion step in a plant cell, plant or plant part in vivo.

The definitions made above apply mutatis mutandis to the following embodiments

The present invention further relates to a method for generating a plant cell, plant or plant part which produces an increased amount of a metabolite of interest when compared to a control, said method comprising: (a) identifying a metabolic conversion step, the modulation of which increases a metabolite of interest in a plant cell, plant or plant part, by the method of any one of claims 1 to 13; and (b) stably modulating the said metabolic enzymatic conversion step such that the amount of the metabolite of interest is increased in vivo in a plant cell, plant or plant part.

The method for generating a plant cell, plant or plant part of the present invention, preferably, is an in vitro method. Moreover, it may comprise steps in addition to those explicitly mentioned above. For example, further steps may relate, e.g., to introducing a compound modulating the said metabolic conversion step in step b). Moreover, one or more of said steps may be performed by automated equipment. Preferably, the generation of said plant cell does not rely exclusively on natural phenomena such as crossing and selection.

As used herein, the term “stably modulating” relates to modulating as defined herein above over an extended period of time. Preferably, stably modulating relates to modulating a metabolic conversion for at least one week, at least two weeks, at least three weeks, at least four weeks, at least one month, at least two months, at least three months, at least six months, at least one year, or more than one year. This kind of stable modulation can, e.g. be achieved by applying an inhibitor to the plant, which is not removed from metabolism to a significant extent over the said period of time, or by introducing a regulable gene into said plant providing for the intended modulation of the amount of the metabolite of interest and applying an inducer or repressor of said inducible gene to said plant for said period of time. More preferably, stably modulating relates to modulating a metabolic conversion starting at a selected point in time and continuing at least until the plant, plant tissue, plant part, or plant cell is harvested or until the end of the growing season. This kind of stable modulation can, e.g. be achieved by introducing a regulable gene into said plant providing for the intended modulation of the amount of the metabolite of interest and applying an inducer or a repressor of said inducible gene to said plant. It is understood by the skilled artisan that said application of an inducer may have to be repeated in order to maintain induction of the inducible gene and, thereby, the modulation of the metabolite of interest. This kind of modulation can, e.g., also be obtained by introducing a genetic construct into said plant, which can be induced to undergo a genetic rearrangement, wherein said genetic rearrangement produces a modified genetic construct being constitutively active in modulating said metabolite of interest. Most preferably, stably modulating relates to modulating a metabolic conversion in a manner stably inherited over at least two generations. Such stable modulation can, e.g. be achieved by introducing a gene coding for an enzyme modulating the amount of a metabolite of interest or by deleting or mutating a gene coding for an enzyme modulating the amount of a metabolite of interest as described herein above. It is understood that stable modulation according to the present invention can also be achieved by indirect methods as described herein above.

The present invention further relates to a plant cell, plant or plant part obtainable by the method for generating a plant cell, plant or plant part, which produces an increased amount of a metabolite of interest when compared to a control, of the present invention.

The present invention also relates to a device, preferably a data processing device, comprising a data processor having tangibly embedded least one of the algorithms of the invention.

The term “device” as used herein relates to a system of means comprising at least the aforementioned means operatively linked to each other as to allow the identification of at least one candidate metabolic conversion step of the present invention. How to link the means in an operating manner will depend on the type of means included into the device. Preferably, the device is capable of generating an output file containing at least one candidate metabolic step according to the invention identified based on applying said algorithm on the stoichiometric network of the present invention.

The present invention further relates to a data carrier comprising the data defining the stoichiometric network model of the present invention.

As used herein, the term data carrier relates to a physical object comprising the data of the present invention in a form legible, preferably directly or indirectly, to a human or a data processing device. Preferably, data are stored in analogous form; more preferably, data are stored in digital form. Preferably, data are stored electronically or magnetically on the data carrier. It is understood that, preferably, a data carrier is not of any predetermined form or configuration. Preferably, the data carrier is a radio-frequency identification (RFID) chip, a memory chip, a CD or DVD, a hard disk, or the like. It is understood by the skilled person that data may be stored in an encrypted form on the data carrier.

All references cited in this specification are herewith incorporated by reference with respect to their entire disclosure content and the disclosure content specifically mentioned in this specification.

### FIGURE LEGENDS

FIG. 1: Different algorithmic approaches for Growth-coupled Design. Regarding their programmatic approach the above mentioned algorithms can be classified as follows: a) Mathematical approach: Bilevel Optimization problem (i.e. OptKnock and RobustKnock) and b) Evolutionary Approach: Genetic algorithm (i.e. OptGene). Figures are modified from Burgard et al., 2003; Patil et al., 2005.

FIG. 2: Flux maps for selected knock-out mutants. A) Flux distribution map of Lys-2KO-RK. B) Flux distribution map of Lys-3KO-OK. Metabolite abbreviations are explained in Table 4

The following Examples shall merely illustrate the invention. They shall not be construed, whatsoever, to limit the scope of the invention.

### EXAMPLE 1

### Reconstruction of Rice Seed Model

A metabolic model of rice seeds was reconstructed in accordance with the reconstruction procedure stated in (Grafahrend-Belau et al., 2009). This bottom-up approach of metabolic reconstruction is based on rice-specific seed knowledge about precise biomass composition as well as definition of model system boundaries such as uptake and excretion reactions for nutrients and other metabolites. Accordingly, the rice seed model only contains reactions and pathways of primary metabolism that are required for biochemical route from affiliated biochemical compounds to synthesis of all specific biomass precursors. Each participating reaction is characterized by its reaction stoichiometry, compartmental localization and literature evidence verifying the reactions' occurrence in rice or other taxonomical related plants such as maize, wheat or barley. Due to lack of available plant and especially rice specific data the following assumption for the overall modeling process are taken into account:
Each reaction is treated as reversible unless it is explicitly declared as irreversible in literature.
Each individual metabolic component (reaction or metabolite) is assigned to one of the following compartments: extracellular media, cytosol, plastid or mitochondrion. In case, there is no localization information available or this metabolic component appears in another compartment than these mentioned above, it is modelled as cytosolic component.
Multi-enzyme complexes are modelled by one single reaction whose reaction stoichiometry is defined by net reaction of all subunits of this enzyme.

The final metabolic model was functionally tested and verified under different growth conditions and genetic modifications elsewhere.

### EXAMPLE 2

### Constraint-Based Modeling

An existing metabolic reconstruction can be used to assess phenotypic properties and functional states of the model organism by applying methods of constraint-based modeling. Assuming metabolic steady state, the system of mass balance equations derived from a metabolic network of n reactions and m metabolites can be represented as follows:

S·v=0

with

αⱼ≦vⱼ≦βⱼ

where S is the stoichiometric matrix (m×n) and v is a flux vector of n metabolic fluxes, with αᵢ as lower and βᵢ as upper bounds for each vᵢ, respectively. The most common constraint-based method is flux balance analysis that uses the principle of linear programming to solve the system of mass balance equations by defining an objective function and searching the allowable solution space for an optimal flux distribution that maximizes or minimizes the objective function (Savinell and Palsson, 1992). While flux balance analysis is preferred for prediction of wild type flux distributions, the following constraint-based methods were used for perturbed networks (including one or more reaction knock-outs): MOMA (Segre et al., 2003) and ROOM (Shlomi et al., 2005).

The whole model simulation including different constraint-based methods and algorithms was achieved using the COBRA toolbox version 2.0.3 (downloaded at Oct. 26, 2011) which is an opensource bundle of M-scripts for model reconstruction and model analysis (Schellenberger et al., 2011). The commercial mathematical environment Matlab R2011b version 7.13 as well as the commercial solver CPLEX from IBM was used for execution of these COBRA scripts. In addition, the SBML toolbox version 4.0.1 and libSBML version 5.1.0b0 are required to import the metabolic model in SBML file format into Matlab for further analysis. The resulting flux distributions of the rice seed model are visualized using the PathwayExplorer add-on FluxViz.

### EXAMPLE 3

### Growth-Coupled Design

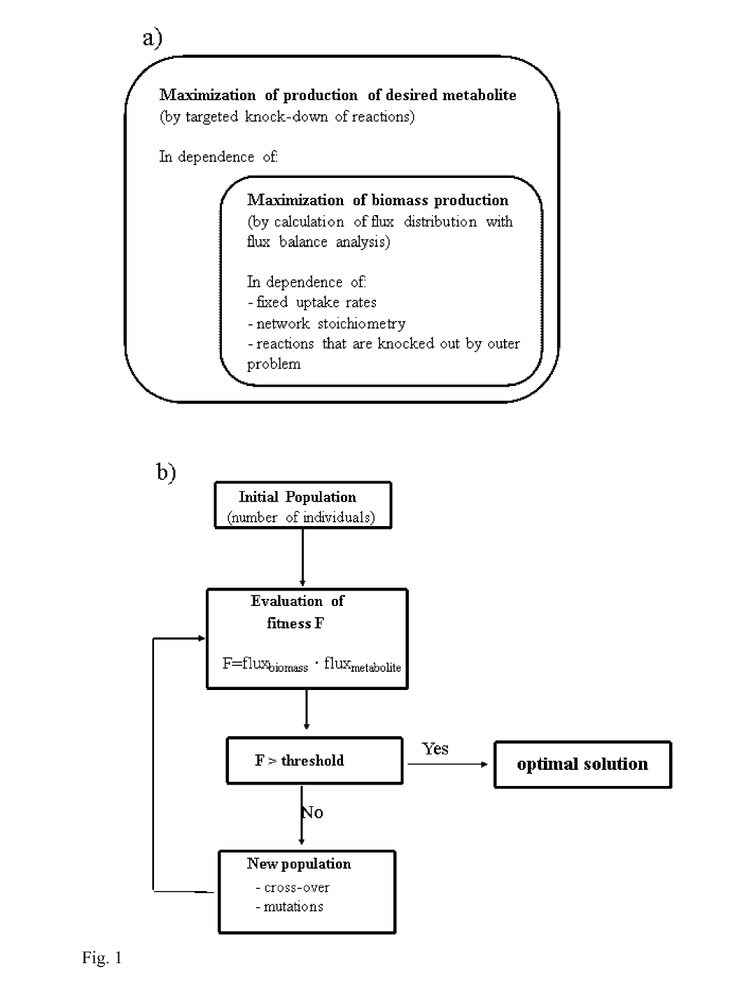

An application of constraint-based modeling is the Growth-coupled Design which is an ‘in-silico’ metabolic engineering strategy coupling metabolite production to growth rate. The following algorithmic approaches of Growth-coupled Design were used to identify knock-out mutants of rice seeds with increased amount of different essential amino acids: the bilevel optimization algorithms OptKnock and RobustKnock, and the genetic algorithm OptGene. Beside the different programmatic approach, all algorithms of Growth-coupled Design provide knock-out mutants characterized by a number of one or more metabolic reactions whose knock-out support production of particular metabolite of interest (FIG. 1).

### EXAMPLE 4

### Predicting Knock-Out Mutants for Rice Seeds with Enhanced Content of Essential Amino Acids

For the purpose of using the Growth-coupled Design to predict knock-out mutants for rice seeds with enhanced content of essential amino acids, the stoichiometric model as well as the corresponding network map needs to be enlarged by the following:
1. Addition of all reactions needed for synthesis of particular essential amino acids, if they are not yet included in the stoichiometric model
2. Addition of (artificial) exchange reaction for particular essential amino acid

The following simulation settings were used for all simulation runs irrespective of the used algorithm:
Uptake rate of sucrose as main carbon source was fixed to 0.014 mmol gDW⁻¹ h⁻¹ (Furbank et al., 2001)
Maximum number of knock-outs is varied between 2 and 4 for OptKnock and RobustKnock, whereas this number was limited to 6 for OptGene
Minimal biomass threshold was fixed to 50% of optimal value (obtained by flux balance analysis under wild type conditions) for OptKnock and RobustKnock
Iterations: OptKnock and RobustKnock were run for each number of allowable knock-outs; OptGene was run for five times

### EXAMPLE 5

### Analysing Enhanced Production of Essential Amino Acids in Rice Seed Metabolism

For the purpose of analysing enhanced production of essential amino acids in rice seed metabolism the following 3 algorithmic approaches for prediction of multiple knock-out mutants were used:
OptKnock,
RobustKnock and
OptGene.

The following essential amino acids were studied in detail: lysine, methionine, cysteine, threonine and tryptophan. Each listed amino acid was analysed using each of the above mentioned algorithms by application of defined simulation settings (see section ‘Experimental Procedures’ for further details). The utilization of similar simulation settings for these approaches allows a general comparison between them regarding their solution quality, their maximum number of knock-outs and their average duration time for one simulation run (see Table 5).

TABLE 5
Evaluation of different algorithms for Growth-coupled Design.
The results of Growth-coupled Design for the 5 essential amino acids
were compared regarding their solution quality, number of feasible
knock-outs and average duration for one simulation run. The property
‘Total’ is a measure how often the different algorithms
found the (best) solution for each amino acid: best solution: 3 pts.,
second-best solution: 2 pts., worst solution: 1 pt. These points were
cumulated in the end.

| Property | OptKnock | RobustKnock | OptGene |
|---|---|---|---|
| Best solution | 1 | 3 | 2 |
| No solution | 2 | 1 | 1 |
| Total | 8 | 12 | 11 |
| Max number of KOs | 5 | 2 | 6 |

| | | |
|---|---|---|
| Duration | Exponential increase | about 30 minutes; |
| | by enhancing the | dependent on number |
| | number of knock-outs | of generations |

By comparing the results of these different algorithms there is no clear preference for one of these algorithms. The both bilevel optimization algorithms OptKnock and RobustKnock are suitable to predict 2-4 knock-outs whereas RobustKnock delivers KO mutants with a higher ranking SSP value in total. In contrast, OptGene can be preferentially used to provide multiple KO mutants with more than 4 knock-outs which are not feasible with the other two algorithms due to the increased mathematical complexity.

### EXAMPLE 6

### Evaluation of KO Mutants

The obtained KO mutants from different simulations of Growth-coupled Design were evaluated by the following ranking criteria (Feist et al., 2010):
1. Product Yield Y_{P}: Maximum amount of product that can be generated by unit of substrate

$${Y}_{P} = \frac{PRODUCTION {RATE}_{PRODUCT}}{COMSUMPTION {RATE}_{SUBSTRATE}} [ \frac{MMOL PRODUCT}{MMOL SUBSTRATE} ]$$

2. Substrate-specific Productivity SSP: Product Yield per unit substrate multiplied by the growth rate

$$SSP = {Y}_{P} \cdot GROWTH RATE [ \frac{MMOL PRODUCT}{MMOL SUBSTRATE \cdot HR} ]$$

For selected knock-out mutants, the overall flux distribution was calculated by the MOMA approach at which the allowable flux through each nominated reaction is set to zero. Finally the main reaction fluxes (flux threshold=1e⁻⁰⁶) are mapped onto the network map using the VANTED add-on FluxMap.

### EXAMPLE 7

### Enhanced Production of Lysine in Rice Seeds

The essential amino acid lysine (chemical formula: C₆H₁₄N₂O₂) belongs to the group of alkaline amino acids such as arginine and histidine. It is synthesized from aspartate through a linear biochemical pathway of 9 enzymes occurring in the plastid. The energy requirements as well as other biochemical intermediates as detailed in Table 6 are required for production of one molecule lysine.

TABLE 6
Biochemical requirements for synthesis of one molecule lysine.
Referring to the net reaction of the synthesis of one molecule lysine,
the listed substrates and products are required and accordingly
provided for other metabolic processes.

| Functional group | Substrates | Products |
|---|---|---|
| Precursors | L-aspartate | |
| | Pyruvate | |
| Energy metabolites | ATP | ADP + P |
| | 2 NADPH | 2 NADP⁺ + H⁺ |
| Other biochemical | Succinyl-CoA | CoA |
| intermediates | L-glutarate | 2-oxoglutarate |
| | | Succinate + CO₂ |

From a modeling point of view, the construction of knock-out mutants of rice seeds with increased lysine content needs the respective precursors, energy sources and the other required biochemical intermediates in a higher extent in comparison to the wild type. In addition, the accumulation of these lysine relevant biochemical intermediates has to be channeled to the synthesis of lysine by knock-out of key metabolic reactions. Different simulations of Growth-coupled Design deliver a list of several knock-out mutants that are defined by a list of metabolic reactions whose knock-out lead to an increased lysine content while minimal biomass accumulation is ensured. These mutants can be further characterized by their exchange flux values as well as their respective flux distributions. Applying the MOMA approach to each knock-out mutant the overall flux distribution including the exchange flux values is obtained.

Referring to the ‘Substrate-specific Productivity’ as ranking criterion, the 4 best knock-out mutants for enhanced lysine content are selected for further analysis (see Table 7). In that case, the 4 best knock-out mutants were obtained from OptKnock and RobustKnock, the both bilevel optimization algorithms. OptGene has also found several knock-out mutants but with a lower SSP value in comparison to the shown knock-out mutants from the other two algorithmic approaches.

TABLE 7
Exchange reaction rates for different lysine mutants
All exchange reactions for 4 selected lysine mutants are shown. Flux values of all
exchange reactions (except biomass reaction) are given by mmol gDW¹ h⁻¹;
biomass flux rate is given by hr⁻¹. The name of each mutant is a concatenation
of (1) essential amino acid, (2) number of knock-outs and (3) the used algorithmic
approach of Growth-coupled Design. Abbreviations: Lys—lysine; KO—knock-out;
OK—OptKnock; RK—RobustKnock; SSP—Substrate-specific Productivity.

| Exchange | Wild type | Lys-2KO-OK | Lys-3KO-OK | Lys-4KO-OK | Lys-2KO-RK |
|---|---|---|---|---|---|
| Uptake | | | | | |
| Sucrose | 0.0144 | 0.0144 | 0.0144 | 0.0144 | 0.0144 |
| O₂ | 0.0117 | 0.0104 | 0.0108 | 0.0103 | 0.0 |
| H₂S | 0.0002 | 0.0001 | 0.0001 | 0.0001 | 0.0001 |
| Asparagine | 0.0 | 0.0040 | 0.0 | 0.0028 | 0.0073 |
| Glutamine | 0.0024 | 0.0021 | 0.0058 | 0.0037 | 0.0 |
| Secretion | | | | | |
| Biomass | 0.0049 | 0.0019 | 0.0019 | 0.0019 | 0.0019 |
| CO₂ | 0.0168 | 0.0296 | 0.0288 | 0.0297 | 0.0246 |
| Lactate | 0.0 | 0.0185 | 0.0199 | 0.0187 | 0.0182 |
| Ethanol | 0.0 | 0.0080 | 0.0092 | 0.0081 | 0.0094 |
| Lysine | 0.0 | 0.0052 | 0.0049 | 0.0056 | 0.0064 |
| SSP | | 6.86e⁻⁰⁴ | 6.46e⁻⁰⁴ | 7.39e⁻⁰⁴ | 8.44e⁻⁰⁴ |
| Ranking | | 3. | 4. | 2. | 1. |

The exchange flux values of a mutant as a first measure describes the similarity of the model borders between knock-out mutant and wild type. Except the sucrose uptake and the minimal biomass threshold which is fixed in all simulations, the remaining exchange flux values vary between the wild type and the different mutants. Oxygen uptake is decreased in all mutants compared to the wild type which in turn activates the fermentation process by producing lactate and ethanol. The uptake fluxes of both nitrogen sources asparagine and glutamine is varied a lot between the different mutants. Two of them (Lys-2KO-OK and Lys-4KO-OK) need both amino acids while the other two mutants just need one of them in order to ensure sufficient nitrogen availability for the metabolic processes. The high amount of produced CO₂ which is doubled compared to the wild type, is not surprising due to the fact that CO₂ is a by-product of lysine synthesis (see Table 6).

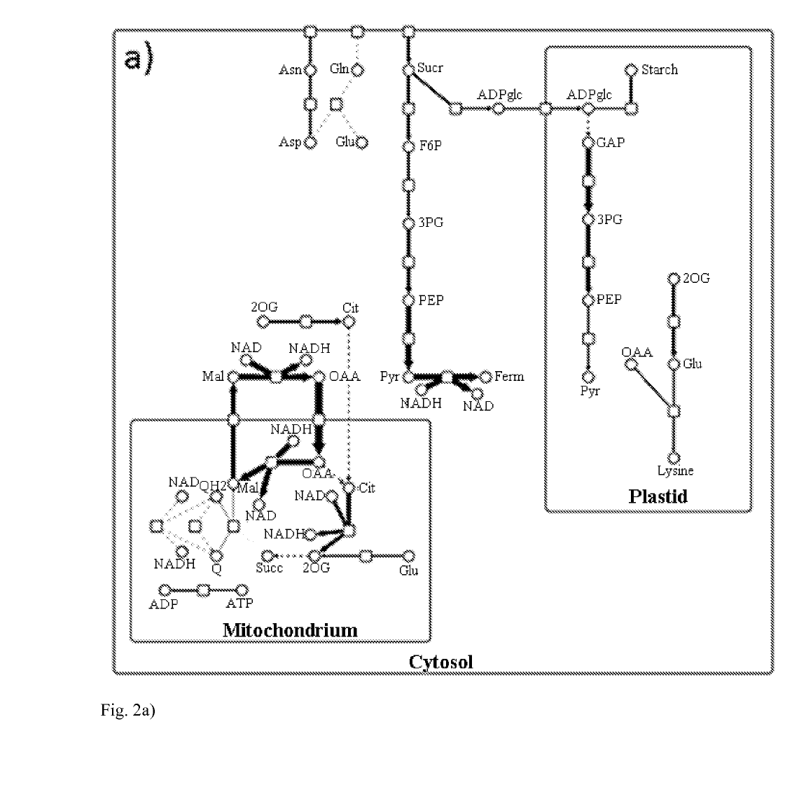

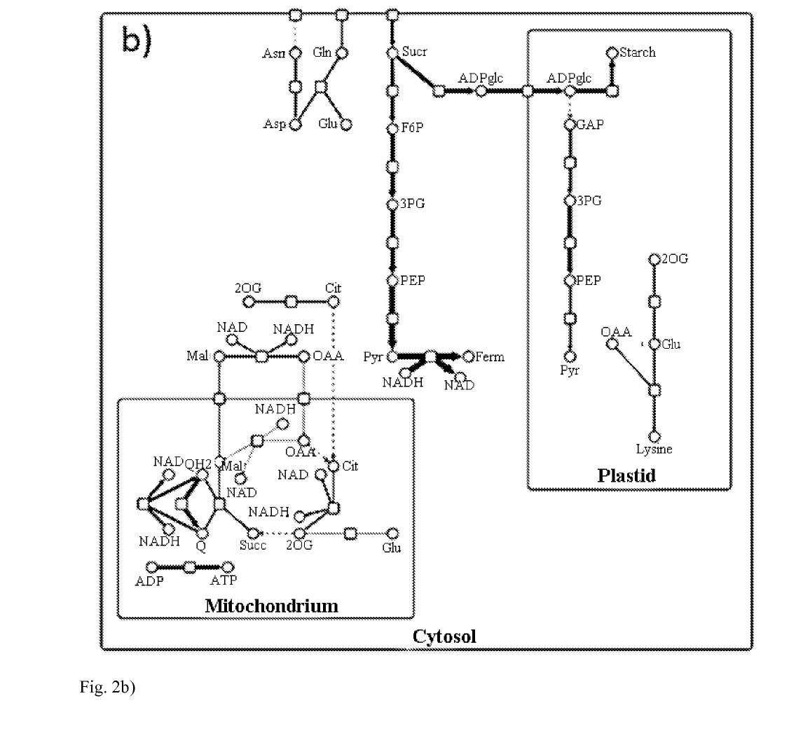

A more comprehensive understanding of the different knock-out mutants can be achieved by generating the corresponding flux maps of each mutant. These maps contain all internal reaction fluxes in addition to the exchange fluxes (see Table 4). The flux value is indicated by width of the reaction arrow, i.e. a high reaction flux value is represented as a thick reaction arrow and vice versa. In the following the flux distribution maps are shown for two selected mutants: Lys2KO-RK and Lys-3KO-OK (see FIG. 2). Referring to the exchange fluxes, these two mutants are very different from each other with respect to their oxygen uptake and their used nitrogen source. Table 8 highlights for each mutant the metabolic reactions whose knock-out was predicted by the respective algorithms of Growth-coupled Design.

TABLE 8
Details for Lys-2KO-RK and Lys-3KO-OK.
These two knock-out mutants are characterized by a number of metabolic
reactions whose knock-out lead to an increase in lysine content in rice
seed metabolism. The metabolic reactions are given by their common
names, EC numbers and their corresponding reaction stoichiometry.
Lys-2KO-RK

| | | | |
|---|---|---|---|
| 1 | Phosphoglycerate kinase | EC | 13BPG[c] + ADP[c] <==> |
| | | 2.7.2.3 | 3PG[c] + ATP[c] |
| 2 | Cytochrome-c oxidase | EC | QH2[m] + 0.5 O2[m] ==> |
| | | 1.9.3.1 | Q[m] + 2 H[m] |

Lys-3KO-OK

| | | | |
|---|---|---|---|
| 1 | NAD+-dependent aldehyde | EC | AcAl[c] + NAD+[c] ==> |
| | dehydrogenase | 1.2.1.3 | AcA[c] + NADH[c] |
| 2 | Fructose-1,6-bisphosphatase | EC | F16BP[c] ==> F6P[c] + |
| | | 3.1.3.11 | P[c] |
| 3 | Asparagine uptake | — | ==>Asn[c] |

By comparing both flux distribution maps, some main differences of flux channeling can be observed. At first, main carbon flux enters the rice seed via the sucrose transporter and is channeled through the sucrose breakdown pathway in both mutants. From there, one portion of the flux is directed to synthesis of ADP-glucose which is transported into the plastid and is the main precursor of starch. The other portion of the main flux enters the glycolysis which produces pyruvate, an important precursor of lysine, in the end. While the Lys-2KO-RK mutant uses the cytosolic as well as the plastidic part of glycolysis to produce pyruvate, the Lys-3KO-RK mutant uses the plastidic part in a higher extent. In addition, many transporters of glycolytic intermediates between cytosol and plastid are very active in both mutants (not shown in the flux maps). The full amount of produced pyruvate cannot be used solely for lysine synthesis, that's why a great portion is used for production of the fermentative metabolites lactate and ethanol. The other important precursor of lysine is aspartate which is directly synthesized from affiliated asparagine in Lys-2KO-RK, while in the other mutant it is generated from the affiliated glutamine by consuming energy in the form of ATP. Another difference between both flux maps is the flux through the TCA cycle which is actually no ‘real’ cycle in the Lys-2KO-RK mutant. The main function of the TCA cycle in this mutant is the remobilization of NADH from NAD which is used during the production of the fermentative products. The other mutant uses the glycolytic enzyme phosphoglycerate kinase (knock-out reaction in Lys-2KO-RK) for remobilization of NADH, and the TCA cycle shows a minimal cycling flux. Furthermore, the metabolic processes of Lys-3KO-OK require a lot of energy due to the high flux activity of oxidative phosphorylation pathway. In the other mutant, the oxidative phosphorylation is knocked-out by the enzyme cytochrome-c oxidase. However, the Lys-2KO-RK is able to synthesize more lysine from the same amount of sucrose using less energy resources in comparison to Lys-3KO-OK

### REFERENCES

### Examples Section

Burgard A. P., Pharkya P., Maranas C. D. (2003) OptKnock: A bilevel programming framework for identifying gene knockout strategies for microbial strain optimization. Biotechnology and Bioengineering. 84, 647-657
Feist A. M., Zielinski D. C., Orth J. D., Schellenberger J., Herrgard M. J., Palsson B. O. (2010) Model-driven evaluation of the production potential for growth-coupled products of Escherichia coli. Metabolic Engineering. 12, 173-186
Furbank R. T., Scofield G. N., Hirose T., Wang X. D., Patrick J. W., Offler C. E. (2001) Cellular localization and function of a sucrose transporter OsSUT1 in developing rice grains. Australian Journal of Plant Physiology. 28, 1187-1196
Grafahrend-Belau E., Schreiber F., Koschützki D., Junker B. H. (2009) Flux balance analysis of barley seeds: a computational approach to study systemic properties of central metabolism. Plant Physiology. 149, 585-598
Hucka M., Finney A., Sauro H. M., Bolouri H., Doyle J. C., Kitano H. (2003) The systems biology markup language (SBML): a medium for representation and exchange of biochemical network models. Bioinformatics. 19, 524-531
Lun D. S., Rockwell G., Guido N. J., Baym M., Kelner J. A., Berger B., Galagan J. E., Church G. M. (2009) Large-scale identification of genetic design strategies using local search. Molecular systems biology. 5, 296
Patil K. R., Rocha I., Förster J., Nielsen J. (2005) Evolutionary programming as a platform for in silico metabolic engineering. BMC Bioinformatics. 6, 308
Pharkya P., Maranas C. D. (2006) An optimization framework for identifying reaction activation/inhibition or elimination candidates for overproduction in microbial systems. Metabolic Engineering. 8(1), 1-13
Ranganathan S., Suthers P. F., Maranas C. D. (2010) OptForce: An optimization procedure for identifying all genetic manipulations leading to targeted overproductions. PLoS Computational Biology. 6, e1000744
Savinell J. M., Palsson B. O. (1992) Network analysis of intermediary metabolism using linear optimization: 1. Development of mathematical formalism. Journal of Theoretical Biology. 154, 421-454
Schellenberger J., Que R., Fleming R. M. T., Thiele I., Orth J. D., Feist A. M., Zielinski D. C., Bordbar A., Lewis N. E., Rahmanian S., Kang J., Hyduke D. R., Palsson B. O. (2011) Quantitative prediction of cellular metabolism with constraint-based models: the COBRA toolbox v2.0. Nature Protocols. 6, 1290-1307
Segre D., Vitkup D., Church G. M. (2002) Analysis of optimality in natural and perturbed metabolic networks. PNAS. 99, 15112-15117
Shlomi T., Berkman O., Ruppin E. (2005) Regulatory on/off minimization of metabolic flux changes after genetic perturbations. PNAS. 102, 7695-7700
Tepper N. and Shlomi T. (2010) Predicting metabolic engineering knockout strategies for chemical production: accounting for competing pathways. Bioinformatics. 26, 536-543
Yang L., Cluett W. R., Mahadevan R. (2011) EMILiO: A fast algorithm for genome-scale strain design. Metabolic Engineering. 13(3), 272-281

TABLE 2
growth-coupled design approaches

| | | Predictions | | | |
|---|---|---|---|---|---|
| Name | Type | for | Availability | Comments | Reference |
| OptKnock | BO | KO | COBRA toolbox | Prediction of gene deletion strategies leading to | Burgard et al. (2003) Biotechnology |
| | | | | overproduction of chemicals of interest | and Bioengineering. 84(6): 647-657 |
| RobustKnock | BO | KO | Matlab script | Prediction of gene deletion strategies leading to | Tepper and Shlomi (2010) |
| | | | | overproduction of chemicals of interest, by | Bioinformatics. 26(4): 536-543 |
| | | | | accounting for the presence of competing pathways | |
| | | | | in the network model | |
| OptGene | HA | KO | COBRA toolbox/ | Identification of gene deletion strategies for | Patil et al. (2005) BMC |
| | | | OptFlux | optimization of a desired phenotypic objective | Bioinformatics. 6: 308 |
| | | | | function (linear + non-linear) | |
| OptForce | BO | KD/KO | none | Identification of possible engineering interventions | Ranganathan et al. (2010) PLoS |
| | | and OEX | | by classifying reactions whether their flux values | Computational Biology. 6(4): |
| | | | | must increase, decrease or become equal to 0 to meet | e1000744 |
| | | | | a pre-specified overproduction target | |
| EMILiO | BO | KD/KO | none | (1) Identification of a subset of reactions with the | Yang et al. (2011) Metabolic |
| | | and OEX | | potential to improve growth-coupled biochemical | Engineering. 13(3): 272-281 |
| | | | | production if their fluxes are optimized and (2) | |
| | | | | quantitatively predict the optimal flux ranges that | |
| | | | | maximize production | |
| OptReg | BO | KD/KO | none | Extension to OptKnock allowing for up and/or down- | Pharkya and Maranas (2006) |
| | | and OEX | | regulation in addition to gene eliminations to meet | Metabolic Engineering. 8: 1-13 |
| | | | | a bioproduction goal | |
| GDLS | HA | KO | COBRA toolbox | local search approach with multiple search paths to | Lun et al. (2009) Molecular |
| | | | | find a set of locally optimal genetic strategies for | Systems Biology. 5: 296 |
| | | | | knock-out mutants | |

TABLE 3
metabolic conversion steps of the rice model. Letters in parentheses relate to the allocation of
a metabolite to a specific cell compartiment; [c] = cytosol, [m] = mitochondrion, [p] = plastid

| Rxn | | | | | | |
|---|---|---|---|---|---|---|
| name | Rxn description | Formula | Subsystem | Reversible | LB | UB |
| R955 | dihydroxy-acid | DIV[p] ==> OIV[p] | Valine Leucine | 0 | 0 | 1000 |
| | dehydratase (valin | | Isoleucine | | | |
| | synthesis) | | Biosynthesis | | | |
| R933 | aspartate-semialdehyde | AspSA[p] + NADP[p] + P[p] <==> | Glycine Serine | 1 | −1000 | 1000 |
| | dehydrogenase | NADPH[p] + PAsp[p] | Threonine | | | |
| | | | Metabolism | | | |
| R852 | citrate synthase | AcCoA[m] + OAA[m] ==> Cit[m] + CoA[m] | TCA Cycle | 0 | 0 | 1000 |
| R1020 | LeuSPONTANEOUS | IPO[p] ==> CO2[p] + OIC[p] | Valine Leucine | 0 | 0 | 1000 |
| | | | Isoleucine | | | |
| | | | Biosynthesis | | | |
| R915 | imidazoleglycerol- | Gln[p] + PRu_AICARP[p] ==> AICAR[p] + | Histidine Metabolism | 0 | 0 | 1000 |
| | phosphate synthase | Glu[p] + IGP[p] | | | | |
| R897 | transaldolase | GAP[p] + S7P[p] <==> E4P[p] + F6P[p] | Pentose Phosphate | 1 | −1000 | 1000 |
| | | | Pathway | | | |
| R820 | aldehyde dehydrogenase | AcAI[c] + NAD[c] ==> AcA[c] + NADH[c] | Glycolysis | 0 | 0 | 1000 |
| | (NAD+) (cALDH) | | Gluconeogenesis | | | |
| R883 | inorganic diphosphatase | PP[p] ==> 2 P[p] | Oxidative | 0 | 0 | 1000 |
| | | | Phosphorylation | | | |
| R799 | diphosphate-fructose-6- | F6P[c] + PP[c] <==> F16P[c] + P[c] | Fructose Mannose | 1 | −1000 | 1000 |
| | phosphate 1- | | Metabolism | | | |
| | phosphotransferase | | | | | |
| R805 | phosphopyruvate | 2PG[c] <==> PEP[c] | Glycolysis | 1 | −1000 | 1000 |
| | hydratase (cENOLASE) | | Gluconeogenesis | | | |
| R797 | phosphoglucose | F6P[c] <==> G6P[c] | Glycolysis | 1 | −1000 | 1000 |
| | isomerase (cPGI) | | Gluconeogenesis | | | |
| R899 | 3-deoxy-7- | E4P[p] + PEP[p] ==> DAH7P[p] + P[p] | Phenylalanine | 0 | 0 | 1000 |
| | phosphoheptulonate | | Tyrosine Tryptophan | | | |
| | synthase | | Biosynthesis | | | |
| R825 | alanine transaminase | 2OG[c] + Ala[c] <==> Glu[c] + Pyr[c] | Alanine Aspartate | 1 | −1000 | 1000 |
| | | | Glutamate Metabolism | | | |
| R925 | amino-acid N- | AcCoA[p] + Glu[p] ==> AcGlu[p] + CoA[p] | Arginine Proline | 0 | 0 | 1000 |
| | acetyltransferase | | Metabolism | | | |
| R930 | ornithine | CP[p] + Or[p] <==> Citru[p] + P[p] | Arginine Proline | 1 | −1000 | 1000 |
| | carbamoyltransferase | | Metabolism | | | |
| R876 | glyceraldehyde-3- | GAP[p] + NADP[p] + P[p] <==> 13BPG[p] + | Glycolysis | 1 | −1000 | 1000 |
| | phosphate | NADPH[p] | Gluconeogenesis | | | |
| | dehydrogenase (NADP+) | | | | | |
| | (phosph.) | | | | | |
| R965 | serine O- | AcCoA[p] + Ser[p] ==> AcSer[p] + CoA[p] | Cysteine Methionine | 0 | 0 | 1000 |
| | acetyltransferase | | Metabolism | | | |
| R796 | phosphoglucomutase | G1P[c] <==> G6P[c] | Glycolysis | 1 | −1000 | 1000 |
| | (cPGM) | | Gluconeogenesis | | | |
| R957 | 2-isopropylmalate | AcCoA[p] + OIV[p] ==> 2IPM[p] + CoA[p] | Valine Leucine | 0 | 0 | 1000 |
| | synthase | | Isoleucine | | | |
| | | | Biosynthesis | | | |
| R919 | histidinol dehydrogenase | Hol[p] + 2 NAD[p] ==> His[p] + 2 NADH[p] | Histidine Metabolism | 0 | 0 | 1000 |
| R823 | asparagine synthase | ATP[c] + Asp[c] + Gln[c] ==> AMP[c] + | Alanine Aspartate | 0 | 0 | 1000 |
| | (glutamine-hydrolysing) | Asn[c] + Glu[c] + PP[c] | Glutamate Metabolism | | | |
| R913 | phosphoribosyl-AMP | PR_AMP[p] ==> PR_AICARP[p] | Histidine Metabolism | 0 | 0 | 1000 |
| | cyclohydrolase | | | | | |
| R874 | fructose-bisphosphate | F16P[p] <==> DHAP[p] + GAP[p] | Glycolysis | 1 | −1000 | 1000 |
| | aldolase (pALD) | | Gluconeogenesis | | | |
| R875 | triose phosphate | DHAP[p] <==> GAP[p] | Glycolysis | 1 | −1000 | 1000 |
| | isomerase (pTIM) | | Gluconeogenesis | | | |
| R928 | acetylornithine | 2OG[p] + AcOr[p] <==> AcGluSA[p] + Glu[p] | Lysine Biosynthesis | 1 | −1000 | 1000 |
| | transaminase | | | | | |
| R943 | 2,3,4,5- | SuccCoA[p] + THDPA[p] ==> CoA[p] + | Lysine Biosynthesis | 0 | 0 | 1000 |
| | tetrahydropyridine-2,6- | SuccAH[p] | | | | |
| | dicarboxylate N- | | | | | |
| | succinyltransferase | | | | | |
| R964 | glycine hydroxymethyl- | Gly[p] + METTHF[p] <==> Ser[p] + THF[p] | Glycine Serine | 1 | −1000 | 1000 |
| | transferase (pSHMT) | | Threonine Metabolism | | | |
| R923 | pyrroline-5-carboxylate | NADH[p] + PyrrC[p] <==> NAD[p] + Pro[p] | Arginine Proline | 1 | −1000 | 1000 |
| | reductase | | Metabolism | | | |
| R973 | acetate-CoA ligase | ATP[p] + AcA[p] + CoA[p] ==> AMP[p] + | Glycolysis | 0 | 0 | 1000 |
| | | AcCoA[p] + PP[p] | Gluconeogenesis | | | |
| R892 | phosphogluconate | 6PG[p] + NADP[p] ==> CO2[p] + | Pentose Phosphate | 0 | 0 | 1000 |
| | dehydrogenase | NADPH[p] + Ru5P[p] | Pathway | | | |
| | (decarboxylating) | | | | | |
| | (p6-PGDH) | | | | | |
| R888 | alpha-glucosidase | Malt[p] ==> 2Glc[p] | Galactose Metabolism | 0 | 0 | 1000 |
| R803 | phosphoglycerate kinase | 13BPG[c] + ADP[c] <==> 3PG[c] + ATP[c] | Glycolysis | 1 | −1000 | 1000 |
| | (cPGlyK) | | Gluconeogenesis | | | |
| R798 | 6-phosphofructokinase | ATP[c] + F6P[c] ==> ADP[c] + F16P[c] | Glycolysis | 0 | 0 | 1000 |
| | (cPFK) | | Gluconeogensis | | | |
| R801 | triose phosphate | DHAP[c] <==> GAP[c] | Glycolysis | 1 | −1000 | 1000 |
| | isomerase (cTIM) | | Gluconeogenesis | | | |
| R922 | pyrroline-5-carboxylate | ATP[p] + Glu[p] + NADPH[p] ==> ADP[p] + | Arginine Proline | 0 | 0 | 1000 |
| | synthase | GluSA[p] + NADP[p] + P[p] | Metabolism | | | |
| R920 | glutamate dehydrogenase | Glu[p] + NADP[p] <==> 2OG[p] + | Alanine Aspartate | 1 | −1000 | 1000 |
| | (NAD(P)) | NADPH[p] + NH3[p] | Glutamate Metabolism | | | |
| R927 | N-acetyl-gamma- | AcGluSA[p] + NADP[p] + P[p] <==> | Arginine Proline | 1 | −1000 | 1000 |
| | glutamyl-phosphate | AcGluP[p] + NADPH[p] | Metabolism | | | |
| | reductase | | | | | |
| R907 | aromatic-amino-acid | 2OG[p] + Agn[p] <==> Glu[p] + PRE[p] | Phenylalanine | 1 | −1000 | 1000 |
| | transaminase | | Tyrosine Tryptophan | | | |
| | (prephenate | | Biosynthesis | | | |
| | aminotransferase) | | | | | |
| R809 | sucrose synthase | UDP[c] + sucrose[c] <==> Frc[c] + | Starch Sucrose | 1 | −1000 | 1000 |
| | | UDPGlc[c] | Metabolism | | | |
| R949 | acetolactate synthase | 2OB[p] + Pyr[p] ==> 2AHB[p] + CO2[p] | Valine Leucine | 0 | 0 | 1000 |
| | (isoleucine synthesis) | | Isoleucine | | | |
| | | | Biosynthesis | | | |
| R929 | aminoacylase | AcOr[p] ==> AcA[p] + Or[p] | Arginine Proline | 0 | 0 | 1000 |
| | | | Metabolism | | | |
| R959 | 3-isopropylmalate | 3IPM[p] + NAD[p] ==> IPO[p] + NADH[p] | Valine Leucine | 0 | 0 | 1000 |
| | dehydrogenase | | Isoleucine | | | |
| | | | Biosynthesis | | | |
| R884 | ADPglucose | ATP[p] + G1P[p] <==> ADPglc[p] + PP[p] | Starch Sucrose | 1 | −1000 | 1000 |
| | pyrophosphorylase | | Metabolism | | | |
| | (pAGPase) | | | | | |
| R811 | sucrose phosphate | F6P[c] + UDPGlc[c] <==> S6P[c] + UDP[c] | Starch Sucrose | 1 | −1000 | 1000 |
| | synthase | | Metabolism | | | |
| R937 | cystathionine gamma- | Cys[p] + PHOMOSer[p] ==> CysTh[p] + P[p] | Cysteine Methionine | 0 | 0 | 1000 |
| | synthase | | Metabolism | | | |
| R835 | argininosuccinate lyase | ArgSucc[c] <==> Arg[c] + Fum[c] | Alanine Aspartate | 1 | −1000 | 1000 |
| | | | Glutamate Metabolism | | | |
| R900 | 3-dehydroquinate | DAH7P[p] ==> 3DHQ[p] + P[p] | Phenylalanine | 0 | 0 | 1000 |
| | synthase | | Tyrosine Tryptophan | | | |
| | | | Biosynthesis | | | |
| R902 | shikimate | NADP[p] + Sh[p] <==> 3DSh[p] + NADPH[p] | Phenylalanine | 1 | −1000 | 1000 |
| | dehydrogenase | | Tyrosine Tryptophan | | | |
| | | | Biosynthesis | | | |
| R950 | ketol-acid | 2AHB[p] + NADPH[p] ==> DMV[p] + | Valine Leucine | 0 | 0 | 1000 |
| | reductoisomerase | NADP[p] | Isoleucine | | | |
| | (isoleucine | | Biosynthesis | | | |
| | synthesis) | | | | | |
| R901 | 3-dehydroquinate | 3DHQ[p] <==> 3DSh[p] | Phenylalanine | 1 | −1000 | 1000 |
| | dehydratase | | Tyrosine Tryptophan | | | |
| | | | Biosynthesis | | | |
| R859 | fumarate hydratase | Mal[m] <==> Fum[m] | TCA Cycle | 1 | −1000 | 1000 |
| R944 | succinyldiaminopimelate | 2OG[p] + SuccDAH[p] <==> Glu[p] + | Lysine Biosynthesis | 1 | −1000 | 1000 |
| | transaminase | SuccAH[p] | | | | |
| R932 | aspartate kinase | ATP[p] + Asp[p] <==> ADP[p] + PAsp[p] | Glycine Serine | 1 | −1000 | 1000 |
| | | | Threonine Metabolism | | | |
| R946 | Diaminopimelate | DAH[p] <==> mDAH[p] | Lysine Biosynthesis | 1 | −1000 | 1000 |
| | epimerase | | | | | |
| R885 | starch synthase (simpl.) | ADPglc[p] ==> ADP[p] + starch[p] | Starch Sucrose | 0 | 0 | 1000 |
| | | | Metabolism | | | |
| R958 | 3-isopropylmalate | 3IPM[p] <==> 2IPM[p] | Valine Leucine | 1 | −1000 | 1000 |
| | dehydratase | | Isoleucine | | | |
| | | | Biosynthesis | | | |
| R880 | pyruvate kinase (pPK) | ADP[p] + PEP[p] ==> ATP[p] + Pyr[p] | Glycolysis | 0 | 0 | 1000 |
| | | | Gluconeogenesis | | | |
| R891 | 6- | GL6P[p] ==> 6PG[p] | Pentose Phosphate | 0 | 0 | 1000 |
| | phosphogluconolactonase | | Pathway | | | |
| R810 | sucrose phosphate | S6P[c] ==> P[c] + sucrose[c] | Starch Sucrose | 0 | 0 | 1000 |
| | phosphatase | | Metabolism | | | |
| R802 | glyceraldehyde-3- | GAP[c] + NAD[c] + P[c] <==> 13BPG[c] + | Glycolysis | 1 | −1000 | 1000 |
| | phosphate dehydrogenase | NADH[c] | Gluconeogenesis | | | |
| | (phosph.) | | | | | |
| R812 | hexokinase | ATP[c] + Glc[c] ==> ADP[c] + G6P[c] | Glycolysis | 0 | 0 | 1000 |
| | | | Gluconeogenesis | | | |
| R893 | ribulose-phosphate | Ru5P[p] <==> X5P[p] | Pentose Phosphate | 1 | −1000 | 1000 |
| | 3-epimerase | | Pathway | | | |
| | (pRuPepimerase) | | | | | |
| R861 | glutamate dehydrogenase | Glu[m] + NAD[m] <==> 2OG[m] + NADH[m] + | Alanine Aspartate | 1 | −1000 | 1000 |
| | | NH3[m] | Glutamate Metabolism | | | |
| R947 | Diaminopimelate | mDAH[p] ==> CO2[p] + Lys[p] | Lysine Biosynthesis | 0 | 0 | 1000 |
| | decarboxylase | | | | | |
| R853 | aconitate hydratase | Cit[m] <==> Icit[m] | TCA Cycle | 1 | −1000 | 1000 |
| | (mACO) | | | | | |
| R833 | glyceraldehyde-3- | GAP[c] + NADP[c] ==> 3PG[c] + NADPH[c] | Glycolysis | 0 | 0 | 1000 |
| | phosphate dehydrogenase | | Gluconeogenesis | | | |
| | (NADP) | | | | | |
| R894 | ribose-5-phosphate | R5P[p] <==> Ru5P[p] | Pentose Phosphate | 1 | −1000 | 1000 |
| | isomerase (pR5P | | Pathway | | | |
| | isomerase) | | | | | |
| R963 | phosphoserine phosphatase | Pser[p] ==> P[p] + Ser[p] | Glycine Serine | 0 | 0 | 1000 |
| | | | Threonine Metabolism | | | |
| R824 | asparaginase | Asn[c] ==> Asp[c] + NH3[c] | Alanine Aspartate | 0 | 0 | 1000 |
| | | | Glutamate Metabolism | | | |
| R966 | cysteine synthase | AcSer[p] + H2S[p] ==> AcA[p] + Cys[p] | Cysteine Methionine | 0 | 0 | 1000 |
| | | | Metabolism | | | |
| R917 | histidinol-phosphate | Glu[p] + IAP[p] ==> 2OG[p] + HolP[p] | Histidine Metabolism | 0 | 0 | 1000 |
| | transaminase | | | | | |
| R829 | isocitrate dehydrogenase | Icit[c] + NADP[c] <==> 2OG[c] + CO2[c] + | TCA Cycle | 1 | −1000 | 1000 |
| | (NADP+)(cICDH) | NADPH[c] | | | | |
| R961 | phosphoglycerate | 3PG[p] + NAD[p] ==> NADH[p] + PHPyr[p] | Glycine Serine | 0 | 0 | 1000 |
| | dehydrogenase | | Threonine Metabolism | | | |
| R872 | phosphoglucose isomerase | F6P[p] <==> G6P[p] | Glycolysis | 1 | −1000 | 1000 |
| | (pPGI) | | Gluconeogenesis | | | |
| R854 | isocitrate dehydrogenase | Icit[m] + NADP[m] <==> 2OG[m] + CO2[m] + | TCA Cycle | 1 | −1000 | 1000 |
| | (NADP+)(mICDH) | NADPH[m] | | | | |
| R938 | cystathionine beta-lyase | CysTh[p] ==> HOMOCys[p] + NH3[p] + | Cysteine Methionine | 0 | 0 | 1000 |
| | | Pyr[p] | Metabolism | | | |
| R814 | UDPglucose | G1P[c] + UTP[c] <==> PP[c] + UDPGlc[c] | Starch Sucrose | 1 | −1000 | 1000 |
| | pyrophosphorylase | | Metabolism | | | |
| R908 | arogenate dehydrogenase | Agn[p] + NAD[p] ==> CO2[p] + NADH[p] + | Phenylalanine | 0 | 0 | 1000 |
| | | Tyr[p] | Tyrosine Tryptophan | | | |
| | | | Biosynthesis | | | |
| R890 | glucose-6-phosphate | G6P[p] + NADP[p] <==> GL6P[p] + | Pentose Phosphate | 1 | −1000 | 1000 |
| | dehydrogenase (p2- | NADPH[p] | Pathway | | | |
| | G6PDH) | | | | | |
| R895 | transketolase | GAP[p] + S7P[p] <==> R5P[p] + X5P[p] | Pentose Phosphate | 1 | −1000 | 1000 |
| | (sedoheptulose 7-P - | | Pathway | | | |
| | ribose 5-P) | | | | | |
| R918 | histidinol-phosphatase | HolP[p] ==> Hol[p] + P[p] | Histidine Metabolism | 0 | 0 | 1000 |
| R905 | chorismate synthase | EPSP[p] ==> Ch[p] + P[p] | Phenylalanine | 0 | 0 | 1000 |
| | | | Tyrosine Tryptophan | | | |
| | | | Biosynthesis | | | |
| R954 | ketol-acid | AcLac[p] + NADPH[p] ==> DIV[p] + NADP[p] | Valine Leucine | 0 | 0 | 1000 |
| | reductoisomerase | | Isoleucine | | | |
| | (valin synthesis) | | Biosynthesis | | | |
| R800 | fructose-bisphosphate | F16P[c] <==> DHAP[c] + GAP[c] | Glycolysis | 1 | −1000 | 1000 |
| | aldolase (cALD) | | Gluconeogenesis | | | |
| R822 | glutamate-ammonia | ATP[c] + Glu[c] + NH3[c] ==> ADP[c] + | Alanine Aspartate | 0 | 0 | 1000 |
| | ligase (cGS, GSI) | Gln[c] + P[c] | Glutamate Metabolism | | | |
| R975 | glutamate synthase | 2OG[p] + Gln[p] + NADH[p] ==> 2 Glu[p] + | Alanine Aspartate | 0 | 0 | 1000 |
| | (NADH) | NAD[p] | Glutamate Metabolism | | | |
| R974 | Proline biosynthesis: | GluSA[p] ==> PyrrC[p] | Arginine Proline | 0 | 0 | 1000 |
| | glutamate 5- | | Metabolism | | | |
| | semialdehyde-1- | | | | | |
| | pyrroline-5-carboxylate | | | | | |
| | (spontaneous reaction) | | | | | |
| R926 | acetylglutamate kinase | ATP[p] + AcGlu[p] ==> ADP[p] + AcGluP[p] | Arginine Proline | 0 | 0 | 1000 |
| | | | Metabolism | | | |
| R806 | pyruvate kinase (cPK) | ADP[c] + PEP[c] ==> ATP[c] + Pyr[c] | Glycolysis | 0 | 0 | 1000 |
| | | | Gluconeogenesis | | | |
| R858 | succinate dehydrogenase | Q[m] + Succ[m] <==> Fum[m] + QH2[m] | TCA Cycle | 1 | −1000 | 1000 |
| | (ubiquinone) | | | | | |
| R906 | chorismate mutase | Ch[p] ==> PRE[p] | Phenylalanine | 0 | 0 | 1000 |
| | | | Tyrosine Tryptophan | | | |
| | | | Biosynthesis | | | |
| R813 | fructokinase | ATP[c] + Frc[c] ==> ADP[c] + F6P[c] | Fructose Mannose | 0 | 0 | 1000 |
| | | | Metabolism | | | |
| R857 | succinate-CoA ligase | ATP[m] + CoA[m] + Succ[m] <==> ADP[m] + | TCA Cycle | 1 | −1000 | 1000 |
| | (ADP-forming) | P[m] + SuccCoA[m] | | | | |
| R952 | branched-chain-amino- | 2OG[p] + Ile[p] <==> Glu[p] + OMV[p] | Valine Leucine | 1 | −1000 | 1000 |
| | acid transaminase | | Isoleucine | | | |
| | (isoleucine synthesis) | | Biosynthesis | | | |
| R889 | adenylate kinase (pAdK) | AMP[p] + ATP[p] <==> 2 ADP[p] | Purine Metabolism | 1 | −1000 | 1000 |
| R904 | 3-phosphoshikimate 1- | PEP[p] + Sh3P[p] <==> EPSP[p] + P[p] | Phenylalanine | 1 | −1000 | 1000 |
| | carboxyvinyltransferase | | Tyrosine Tryptophan | | | |
| | | | Biosynthesis | | | |
| R830 | malate dehydrogenase | Mal[c] + NAD[c] <==> NADH[c] + OAA[c] | TCA Cycle | 1 | −1000 | 1000 |
| | (cMalDH) | | | | | |
| R816 | nucleoside-diphosphate | ATP[c] + UDP[c] <==> ADP[c] + UTP[c] | Purine Metabolism | 1 | −1000 | 1000 |
| | kinase (cNDPkin: UDP) | | | | | |
| R909 | arogenate dehydratase | Agn[p] ==> CO2[p] + Phe[p] | Phenylalanine | 0 | 0 | 1000 |
| | | | Tyrosine Tryptophan | | | |
| | | | Biosynthesis | | | |
| R819 | lactate dehydrogenase | Lac[c] + NAD[c] <==> NADH[c] + Pyr[c] | Glycolysis | 1 | −1000 | 1000 |
| | | | Gluconeogenesis | | | |
| R936 | threonine synthase | PHOMOSer[p] ==> P[p] + Thr[p] | Glycine Serine | 0 | 0 | 1000 |
| | | | Threonine Metabolism | | | |
| R834 | argininosuccinate | ATP[c] + Asp[c] + Citru[c] <==> AMP[c] + | Alanine Aspartate | 1 | −1000 | 1000 |
| | synthase | ArgSucc[c] + PP[c] | Glutamate Metabolism | | | |
| R951 | dihydroxy-acid dehydratase | DMV[p] ==> OMV[p] | Valine Leucine | 0 | 0 | 1000 |
| | (isoleucine synthesis) | | Isoleucine | | | |
| | | | Biosynthesis | | | |
| R856 | oxoglutarate | 2OG[m] + CoA[m] + NAD[m] ==> CO2[m] + | TCA Cycle | 0 | 0 | 1000 |
| | dehydrogenase (succinyl- | NADH[m] + SuccCoA[m] | | | | |
| | transferring) | | | | | |
| R914 | 1-(5-phosphoribosyl)-5- | PR_AICARP[p] ==> PRu_AICARP[p] | Histidine Metabolism | 0 | 0 | 1000 |
| | ((5-phosphoribosyl- | | | | | |
| | amino)methylidene- | | | | | |
| | amino)imid azole-4-carboxamide | | | | | |
| | isomerase | | | | | |
| R953 | acetolactate synthase | 2 Pyr[p] ==> AcLac[p] + CO2[p] | Valine Leucine | 0 | 0 | 1000 |
| | (valin synthesis) | | Isoleucine | | | |
| | | | Biosynthesis | | | |
| R873 | 6-phosphofructokinase | ATP[p] + F6P[p] ==> ADP[p] + F16P[p] | Glycolysis | 0 | 0 | 1000 |
| | (pPFK) | | Gluconeogenesis | | | |
| R862 | aspartate transaminase | 2OG[m] + Asp[m] <==> Glu[m] + OAA[m] | Alanine Aspartate | 1 | −1000 | 1000 |
| | (mAAT) | | Glutamate Metabolism | | | |
| R863 | malate dehydrogenase | Mal[m] + NAD[m] ==> CO2[m] + NADH[m] + | Pyruvate Metabolism | 0 | 0 | 1000 |
| | (decarboxylating) | Pyr[m] | | | | |
| R896 | transketolase (fructose | F6P[p] + GAP[p] <==> E4P[p] + X5P[p] | Pentose Phosphate | 1 | −1000 | 1000 |
| | 6-P - erythrose 4-P) | | Pathway | | | |
| R912 | phosphoribosyl-ATP | PR_ATP[p] ==> PP[p] + PR_AMP[p] | Histidine Metabolism | 0 | 0 | 1000 |
| | diphosphatase | | | | | |
| R821 | aspartate transaminase | 2OG[c] + Asp[c] <==> Glu[c] + OAA[c] | Alanine Aspartate | 1 | −1000 | 1000 |
| | (cAAT) | | Glutamate Metabolism | | | |
| R851 | pyruvate dehydrogenase | CoA[m] + NAD[m] + Pyr[m] ==> AcCoA[m] + | Glycolysis | 0 | 0 | 1000 |
| | complex (mPyrDH) | CO2[m] + NADH[m] | Gluconeogenesis | | | |
| R804 | phosphoglycerate mutase | 3PG[c] <==> 2PG[c] | Glycolysis | 1 | −1000 | 1000 |
| | (cPGlyM) | | Gluconeogenesis | | | |
| R931 | carbamoyl-phosphate | 2 ATP[p] + CO2[p] + Gln[p] ==> 2 ADP[p] + | Arginine Proline | 0 | 0 | 1000 |
| | synthase (glutamine- | CP[p] + Glu[p] + P[p] | Metabolism | | | |
| | hydrolysing) | | | | | |
| R832 | phosphoenolpyruvate | ATP[c] + OAA[c] ==> ADP[c] + CO2[c] + | Glycolysis | 0 | 0 | 1000 |
| | carboxykinase (ATP) | PEP[c] | Gluconeogenesis | | | |
| R878 | phosphoglycerate mutase | 3PG[p] <==> 2PG[p] | Glycolysis | 1 | −1000 | 1000 |
| | (pPGlyM) | | Gluconeogenesis | | | |
| R934 | homoserine | HOMOSer[p] + NAD[p] <==> AspSA[p] + | Glycine Serine | 1 | −1000 | 1000 |
| | dehydrogenase | NADH[p] | Threonine Metabolism | | | |
| R910 | ribose-phosphate | ATP[p] + R5P[p] <==> AMP[p] + PRPP[p] | Pentose Phosphate | 1 | −1000 | 1000 |
| | diphosphokinase | | Pathway | | | |
| | (pPRPPS) | | | | | |
| R817 | pyruvate decarboxylase | Pyr[c] ==> AcAl[c] + CO2[c] | Glycolysis | 0 | 0 | 1000 |
| | | | Gluconeogenesis | | | |
| R911 | ATP phosphoribosyl- | ATP[p] + PRPP[p] ==> PP[p] + PR_ATP[p] | Histidine Metabolism | 0 | 0 | 1000 |
| | transferase | | | | | |
| R815 | ADPglucose | ATP[c] + G1P[c] <==> ADPglc[c] + PP[c] | Starch Sucrose | 1 | −1000 | 1000 |
| | pyrophosphorylase | | Metabolism | | | |
| | (cAGPase) | | | | | |
| R871 | phosphoglucomutase | G1P[p] <==> G6P[p] | Glycolysis | 1 | −1000 | 1000 |
| | (pPGM) | | Gluconeogenesis | | | |
| R831 | phosphoenolpyruvate | CO2[c] + PEP[c] ==> OAA[c] + P[c] | Pyruvate Metabolism | 0 | 0 | 1000 |
| | carboxylase | | | | | |
| R860 | malate dehydrogenase | Mal[m] + NAD[m] <==> NADH[m] + OAA[m] | TCA Cycle | 1 | −1000 | 1000 |
| | (mMalDH) | | | | | |
| R879 | phosphopyruvate hydratase | 2PG[p] <==> PEP[p] | Glycolysis | 1 | −1000 | 1000 |
| | (pENOLASE) | | Gluconeogenesis | | | |
| R887 | beta-amylase (modell) | 2 starch[p] ==> Malt[p] | Starch Sucrose | 0 | 0 | 1000 |
| | | | Metabolism | | | |
| R877 | phosphoglycerate kinase | 13BPG[p] + ADP[p] <==> 3PG[p] + ATP[p] | Glycolysis | 1 | −1000 | 1000 |
| | (pPGlyK) | | Gluconeogenesis | | | |
| R886 | alpha-amylase (modell) | 3 starch[p] ==> Glc[p] + Malt[p] | Starch Sucrose | 0 | 0 | 1000 |
| | | | Metabolism | | | |
| R945 | succinyl-diaminopimelate | SuccDAH[p] ==> DAH[p] + Succ[p] | Lysine Biosynthesis | 0 | 0 | 1000 |
| | desuccinylase | | | | | |
| R818 | alcohol dehydrogenase | Eth[c] + NAD[c] <==> AcAl[c] + NADH[c] | Glycolysis | 1 | −1000 | 1000 |
| | | | Gluconeogensis | | | |
| R903 | shikimate kinase | ATP[p] + Sh[p] ==> ADP[p] + Sh3P[p] | Phenylalanine | 0 | 0 | 1000 |
| | | | Tyrosine Tryptophan | | | |
| | | | Biosynthesis | | | |
| R916 | imidazoleglycerol- | IGP[p] ==> IAP[p] | Histidine Metabolism | 0 | 0 | 1000 |
| | phosphate dehydratase | | | | | |
| R942 | dihydrodipicolinate | NADP[p] + THDPA[p] <==> DPA[p] + | Lysine Biosynthesis | 1 | −1000 | 1000 |
| | reductase | NADPH[p] | | | | |
| R935 | homoserine kinase | ATP[p] + HOMOSer[p] ==> ADP[p] + | Glycine Serine | 0 | 0 | 1000 |
| | | PHOMOSer[p] | Threonine Metabolism | | | |
| R956 | branched-chain-amino- | 2OG[p] + Val[p] <==> Glu[p] + OIV[p] | Valine Leucine | 1 | −1000 | 1000 |
| | acid transaminase | | Isoleucine | | | |
| | (valine synthesis) | | Biosynthesis | | | |
| R827 | adenylate kinase (cAdK) | AMP[c] + ATP[c] <==> 2 ADP[c] | Purine Metabolism | 1 | −1000 | 1000 |
| R948 | threonine ammonia-lyase | Thr[p] ==> 2OB[p] + NH3[p] | Glycine Serine | 0 | 0 | 1000 |
| | | | Threonine Metabolism | | | |
| R941 | dihydrodipicolinate | AspSA[p] + Pyr[p] ==> DPA[p] | Lysine Biosynthesis | 0 | 0 | 1000 |
| | synthase | | | | | |
| R924 | ornithine-oxo-acid | 2OG[p] + Or[p] <==> GluSA[p] + Glu[p] | Arginine Proline | 1 | −1000 | 1000 |
| | transaminase | | Metabolism | | | |
| R837 | glutamate decarboxylase | Glu[c] ==> CO2[c] + Gaba[c] | Alanine Aspartate | 0 | 0 | 1000 |
| | | | Glutamate Metabolism | | | |
| R865 | 4-aminobutyrate | 2OG[m] + Gaba[m] <==> Glu[m] + | Alanine Aspartate | 1 | −1000 | 1000 |
| | transaminase | SuccSAl[m] | Glutamate Metabolism | | | |
| R866 | succinate-semialdehyde | NADP[m] + SuccSAl[m] ==> NADPH[m] + | Alanine Aspartate | 0 | 0 | 1000 |
| | dehydrogenase | Succ[m] | Glutamate Metabolism | | | |
| | (NAD(P)+) | | | | | |
| R881 | fructose-1,6- | F16P[p] ==> F6P[p] + P[p] | Glycolysis | 0 | 0 | 1000 |
| | bisphosphatase | | Gluconeogenesis | | | |
| | (pFBPase) | | | | | |
| R808 | pyruvate, phosphate | ATP[c] + P[c] + Pyr[c] <==> AMP[c] + | Pyruvate Metabolism | 1 | −1000 | 1000 |
| | dikinase (cPPDK) | PEP[c] + PP[c] | | | | |
| R807 | fructose-1,6- | F16P[c] ==> F6P[c] + P[c] | Glycolysis | 0 | 0 | 1000 |
| | bisphosphatase | | Gluconeogenesis | | | |
| | (cFBPase) | | | | | |
| R855 | isocitrate dehydrogenase | Icit[m] + NAD[m] <==> 2OG[m] + CO2[m] + | TCA Cycle | 1 | −1000 | 1000 |
| | (NAD+) (mlCDH) | NADH[m] | | | | |
| R870 | H+-exporting ATPase | ADP[m] + 3 Hext + P[m] <==> ATP[m] | Oxidative | 1 | −1000 | 1000 |
| | | | Phosphorylation | | | |
| R962 | phosphoserine | Glu[p] + PHPyr[p] ==> 2OG[p] + Pser[p] | Glycine Serine | 0 | 0 | 1000 |
| | transaminase | | Threonine Metabolism | | | |
| R972 | malate dehydrogenase | Mal[p] + NADP[p] ==> CO2[p] + NADPH[p] + | Pyruvate Metabolism | 0 | 0 | 1000 |
| | (oxaloacetate- | Pyr[p] | | | | |
| | decarboxylating) | | | | | |
| | (NADP+) | | | | | |
| R939 | methionine synthase | HOMOCys[p] + MTHF[p] ==> Met[p] + | Cysteine Methionine | 0 | 0 | 1000 |
| | (pMS) | THF[p] | Metabolism | | | |
| R967 | phosphoribosylamino- | AICAR[p] + FTHF[p] <==> PRFICA[p] + | Purine Metabolism | 1 | −1000 | 1000 |
| | imidazolecarboxamide | THF[p] | | | | |
| | formyltransferase | | | | | |
| R971 | nucleoside-diphosphate | ATP[p] + GDP[p] <==> ADP[p] + GTP[p] | Purine Metabolism | 1 | −1000 | 1000 |
| | kinase (pNDPkin: GDP) | | | | | |
| R969 | adenylosuccinate synthase | Asp[p] + GTP[p] + IMP[p] ==> Asuc[p] + | Purine Metabolism | 0 | 0 | 1000 |
| | | GDP[p] + P[p] | | | | |
| R968 | IMP cyclohydrolase | IMP[p] <==> PRFICA[p] | Purine Metabolism | 1 | −1000 | 1000 |
| R970 | adenylosuccinate lyase | Asuc[p] <==> AMP[p] + Fum[p] | Purine Metabolism | 1 | −1000 | 1000 |
| | (AMP) | | | | | |
| R845 | methenyltetrahydrofolate | METHF[c] <==> FTHF[c] | Folate Metabolism | 1 | −1000 | 1000 |
| | cyclohydrolase | | | | | |
| | (cMTHCH) | | | | | |
| R847 | methylenetetrahydrofolate | METTHF[c] + NADH[c] ==> MTHF[c] + | Folate Metabolism | 0 | 0 | 1000 |
| | reductase (NAD(P)H) | NAD[c] | | | | |
| | (cMTFHR) | | | | | |
| R846 | methylenetetrahydro- | METTHF[c] + NADP[c] <==> METHF[c] + | Folate Metabolism | 1 | −1000 | 1000 |
| | folate dehydrogenase | NADPH[c] | | | | |
| | (NADP+)(cMTHD) | | | | | |
| R844 | formate-tetrahydrofolate | ATP[c] + For[c] + THF[c] <==> ADP[c] + | Folate Metabolism | 1 | −1000 | 1000 |
| | ligase (cFTHFS) | FTHF[c] + P[c] | | | | |
| R867 | NADH dehydrogenase | NADH[m] + Q[m] ==> 2 Hext + NAD[m] + | Oxidative | 0 | 0 | 1000 |
| | (ubiquinone) | QH2[m] | Phosphorylation | | | |
| R869 | cytochrome-c oxidase | 0.5 O2[m] + QH2[m] ==> 2 Hext + Q[m] | Oxidative | 0 | 0 | 1000 |
| | | | Phosphorylation | | | |
| R1004 | adenosine kinase | ADN[c] + ATP[c] ==> ADP[c] + AMP[c] | Purine Metabolism | 0 | 0 | 1000 |
| R1009 | ATP citrate synthase | ATP[p] + Cit[p] + CoA[p] ==> ADP[p] + | TCA Cycle | 0 | 0 | 1000 |
| | | AcCoA[p] + OAA[p] + P[p] | | | | |
| R960 | branced-chain-amino- | 2OG[p] + Leu[p] <==> Glu[p] + OIC[p] | Valine Leucine | 1 | −1000 | 1000 |
| | acid transaminase | | Isoleucine | | | |
| | (leucine synthesis) | | Biosynthesis | | | |
| R1012 | aspartate transaminase | 2OG[p] + Asp[p] <==> Glu[p] + OAA[p] | Alanine Aspartate | 1 | −1000 | 1000 |
| | (pAAT) | | Glutamate Metabolism | | | |
| R1014 | glycine hydroxymethyl- | Gly[m] + METTHF[m] <==> Ser[m] + THF[m] | Glycine Serine | 1 | −1000 | 1000 |
| | transferase (mSHMT) | | Threonine Metabolism | | | |
| R1013 | glycine decarboxylase | Gly[m] + NADH[m] + THF[m] <==> CO2[m] + | Folate Metabolism | 1 | −1000 | 1000 |
| | system | METTHF[m] + NAD[m] + NH3[m] | | | | |
| R1015 | glycine hydroxymethyl- | Gly[c] + METTHF[c] <==> Ser[c] + THF[c] | Glycine Serine | 1 | −1000 | 1000 |
| | transferase (cSHMT) | | Threonine Metabolism | | | |
| R828 | aconitate hydratase | Cit[c] <==> Icit[c] | TCA Cycle | 1 | −1000 | 1000 |
| | (cA-CO) | | | | | |
| R848 | isocitrate lyase | Icit[c] ==> Glx[c] + Succ[c] | Glyoxylate Cycle | 0 | 0 | 1000 |
| R850 | oxalate decarboxylase | Oxl[c] ==> CO2[c] + For[c] | Formate Metabolism | 0 | 0 | 1000 |
| R849 | glyoxylate oxidase | Glx[c] + O2[c] ==> Oxl[c] | Formate Metabolism | 0 | 0 | 1000 |
| R742 | sucrose transporter | Hext <==> sucrose[c] | Uptake | 1 | −1000 | 1000 |
| R746 | pyruvate transporter | Pyr[c] <==> Pyr[m] | Internal Transport | 1 | −1000 | 1000 |
| | (simpl.) | | | | | |
| R747 | glutamate/aspartate | Asp[m] + Glu[c] <==> Asp[c] + Glu[m] | Internal Transport | 1 | −1000 | 1000 |
| | transporter | | | | | |
| R769 | ADP-glucose transporter | ADP[p] + ADPglc[c] <==> ADP[c] + | Internal Transport | 1 | −1000 | 1000 |
| | (AMP) | ADPglc[p] | | | | |
| R743 | AA transporter | Hext <==> Asn[c] | Uptake | 1 | −1000 | 1000 |
| | (asparagine) | | | | | |
| R744 | AA transporter | Hext <==> Gln[c] | Uptake | 1 | −1000 | 1000 |
| | (glutamine) | | | | | |
| R1023 | 6-phosphogluconolactonase | GL6P[c] ==> 6PG[c] | Pentose Phosphate | 0 | 0 | 1000 |
| | | | Pathway | | | |
| R1022 | glucose-6-phosphate | G6P[c] + NADP[c] <==> GL6P[c] + | Pentose Phosphate | 1 | −1000 | 1000 |
| | dehydrogenase (c-G6PDH) | NADPH[c] | Pathway | | | |
| R1025 | ribulose-phosphate 3- | Ru5P[c] <==>X5P[c] | Pentose Phosphate | 1 | −1000 | 1000 |
| | epimerase (cRuPepimerase) | | Pathway | | | |
| R1024 | phosphogluconate | 6PG[c] + NADP[c] ==> CO2[c] + | Pentose Phosphate | 0 | 0 | 1000 |
| | dehydrogenase | NADPH[c] + Ru5P[c] | Pathway | | | |
| | (decarboxylating) | | | | | |
| | (c6-PGDH) | | | | | |
| R999 | CO2export | CO2[c] <==> | Excretion | 1 | −1000 | 1000 |
| R1000 | biomass export | biomass <==> | Excretion | 1 | −1000 | 1000 |
| R745 | O2-diffusion | <==> O2[c] | Uptake | 1 | −1000 | 1000 |
| R770 | G1P transporter | G1P[p] + P[c] <==> G1P[c] + P[p] | Internal Transport | 1 | −1000 | 1000 |
| R774 | glucose transporter | Glc[c] <==> Glc[p] | Internal Transport | 1 | −1000 | 1000 |
| R775 | triosephosphat/P | GAP[p] + P[c] <==> GAP[c] + P[p] | Internal Transport | 1 | −1000 | 1000 |
| | translocator (TPT1 GAP) | | | | | |
| R776 | triosephosphat/P | DHAP[p] + P[c] <==> DHAP[c] + P[p] | Internal Transport | 1 | −1000 | 1000 |
| | translocator (TPT2 DHAP) | | | | | |
| R777 | triosephosphat/P | 3PG[p] + P[c] <==> 3PG[c] + P[p] | Internal Transport | 1 | −1000 | 1000 |
| | translocator (TPT3 3-PGA) | | | | | |
| R778 | phosphoenolpyruvate/ | PEP[p] + P[c] <==> PEP[c] + P[p] | Internal Transport | 1 | −1000 | 1000 |
| | phosphat transporter | | | | | |
| R780 | malate/2OG transporter | 2OG[c] + Mal[p] <==> 2OG[p] + Mal[c] | Internal Transport | 1 | −1000 | 1000 |
| R781 | malate/fumarate | Fum[p] + Mal[c] <==> Fum[c] + Mal[p] | Internal Transport | 1 | −1000 | 1000 |
| | transporter | | | | | |
| R782 | malate/glutamate | Glu[p] + Mal[c] <==> Glu[c] + Mal[p] | Internal Transport | 1 | −1000 | 1000 |
| | transporter | | | | | |
| R783 | malate/aspartate | Asp[p] + Mal[c] <==> Asp[c] + Mal[p] | Internal Transport | 1 | −1000 | 1000 |
| | transporter | | | | | |
| R748 | OAA/malate transporter | Mal[m] + OAA[c] <==> Mal[c] + OAA[m] | Internal Transport | 1 | −1000 | 1000 |
| R749 | OAA/2OG transporter | 2OG[m] + OAA[c] <==> 2OG[c] + OAA[m] | Internal Transport | 1 | −1000 | 1000 |
| R750 | OAA/succinate transporter | OAA[c] + Succ[m] <==> OAA[m] + Succ[c] | Internal Transport | 1 | −1000 | 1000 |
| R751 | OAA/citrate transporter | Cit[m] + OAA[c] <==> Cit[c] + OAA[m] | Internal Transport | 1 | −1000 | 1000 |
| R752 | OAA/aspartate transporter | Asp[m] + OAA[c] <==> Asp[c] + OAA[m] | Internal Transport | 1 | −1000 | 1000 |
| R756 | succinate/malate | Mal[m] + Succ[c] <==> Mal[c] + Succ[m] | Internal Transport | 1 | −1000 | 1000 |
| | transporter | | | | | |
| R755 | succinate/P transporter | P[c] + Succ[m] <==> P[m] + Succ[c] | Internal Transport | 1 | −1000 | 1000 |
| R757 | malate/P transporter | Mal[m] + P[c] <==> Mal[c] + P[m] | Internal Transport | 1 | −1000 | 1000 |
| R758 | 2OG/citrate transporter | 2OG[m] + Cit[c] <==> 2OG[c] + Cit[m] | Internal Transport | 1 | −1000 | 1000 |
| R759 | 2OG/succinate transporter | 2OG[c] + Succ[m] <==> 2OG[m] + Succ[c] | Internal Transport | 1 | −1000 | 1000 |
| R760 | malate/citrate transporter | Cit[c] + Mal[m] <==> Cit[m] + Mal[c] | Internal Transport | 1 | −1000 | 1000 |
| R761 | succinate/citrate | Cit[c] + Succ[m] <==> Cit[m] + Succ[c] | Internal Transport | 1 | −1000 | 1000 |
| | transporter | | | | | |
| R1021 | mal transporter | Mal[p] <==> Mal[c] | Internal Transport | 1 | −1000 | 1000 |
| R1011 | pyruvate transporter (p) | Pyr[c] <==> Pyr[p] | Internal Transport | 1 | −1000 | 1000 |
| R1008 | malate/citrate transporter | Cit[p] + Mal[c] <==> Cit[c] + Mal[p] | Internal Transport | 1 | −1000 | 1000 |
| R1010 | malate/OAA transporter | Mal[c] + OAA[p] <==> Mal[p] + OAA[c] | Internal Transport | 1 | −1000 | 1000 |
| R795 | succinate/fumarate | Fum[m] + Succ[c] <==> Fum[c] + Succ[m] | Internal Transport | 1 | −1000 | 1000 |
| | transporter | | | | | |
| R1027 | invertase | sucrose[c] ==> Frc[c] + Glc[c] | Starch Sucrose | 0 | 0 | 1000 |
| | | | Metabolism | | | |
| R771 | phosphate transporter | P[c] <==> P[p] | Internal Transport | 1 | −1000 | 1000 |
| R996 | ethanol export | Eth[c]<==> | Excretion | 1 | −1000 | 1000 |
| R997 | lactate export | Lac[c]<==> | Excretion | 1 | −1000 | 1000 |
| R993 | H2S diffusion (cm) | <==>H2S[c] | Uptake | 1 | −1000 | 1000 |
| R762 | phosphate transporter | P[m] <==> P[c] | Internal Transport | 1 | −1000 | 1000 |
| R763 | ATP/ADP transporter | ADP[m] + ATP[c] <==> ADP[c] + ATP[m] | Internal Transport | 1 | −1000 | 1000 |
| R764 | GABA/glutamate transporter | Gaba[m] + Glu[c] <==> Gaba[c] + Glu[m] | Internal Transport | 1 | −1000 | 1000 |
| R766 | CO2-diffusion | CO2[c] <==> CO2[m] | Internal Transport | 1 | −1000 | 1000 |
| R767 | O2-diffusion | O2[c] <==> O2[m] | Internal Transport | 1 | −1000 | 1000 |
| R768 | NH3-diffusion | NH3[c] <==> NH3[m] | Internal Transport | 1 | −1000 | 1000 |
| R1016 | AA transporter p (serine) | Ser[c] <==> Ser[m] | Internal Transport | 1 | −1000 | 1000 |
| R1018 | AA transporter m (gly) | Gly[c] <==> Gly[m] | Internal Transport | 1 | −1000 | 1000 |
| R794 | malate/2OG transporter | 2OG[m] + Mal[c] <==> 2OG[c] + Mal[m] | Internal Transport | 1 | −1000 | 1000 |
| R1026 | X5P/P transporter | P[p] + X5P[c] <==> P[c] + X5P[p] | Internal Transport | 1 | −1000 | 1000 |
| R772 | ATP/AD P transporter | ADP[p] + ATP[c] <==> ADP[c] + ATP[p] | Internal Transport | 1 | −1000 | 1000 |
| R994 | H2S diffusion (p) | H2S[c] ==> H2S[p] | Internal Transport | 0 | 0 | 1000 |
| R790 | folate transporter (THF) | THF[c] <==> THF[p] | Internal Transport | 1 | −1000 | 1000 |
| R789 | CO2-diffusion | CO2[c] <==> CO2[p] | Internal Transport | 1 | −1000 | 1000 |
| R785 | AA transporter (glutamine) | Gln[c] <==> Gln[p] | Internal Transport | 1 | −1000 | 1000 |
| R786 | AA transporter (citrulline) | Citru[c] <==> Citru[p] | Internal Transport | 1 | −1000 | 1000 |
| R787 | acetate diffusion | AcA[c] <==>AcA[p] | Internal Transport | 1 | −1000 | 1000 |
| R1019 | AA transporter p (gly) | Gly[p] <==> Gly[c] | Internal Transport | 1 | −1000 | 1000 |
| R1017 | AA transporter m (serine) | Ser[p] <==> Ser[c] | Internal Transport | 1 | −1000 | 1000 |
| R940 | succinate-CoA ligase | ATP[p] + CoA[p] + Succ[p] <==> ADP[p] + | TCA Cycle | 1 | −1000 | 1000 |
| | (ADP-forming) | P[p] + SuccCoA[p] | | | | |
| R791 | folate transporter | MTHF[c] <==> MTHF[p] | Internal Transport | 1 | −1000 | 1000 |
| | (MTHF) | | | | | |
| R793 | folate transporter (FTHF) | FTHF[c] <==> FTHF[p] | Internal Transport | 1 | −1000 | 1000 |
| R792 | folate transporter | METTHF[c] <==> METTHF[p] | Internal Transport | 1 | −1000 | 1000 |
| | (METTHF) | | | | | |
| R788 | NH3S-diffusion | NH3[p] <==> NH3[c] | Internal Transport | 1 | −1000 | 1000 |
| R1028 | anthranilate synthase | Ch[p] + Gln[p] ==> Ant[p] + Glu[p] + Pyr[p] | Phenylalanine | 0 | 0 | 1000 |
| | | | Tyrosine Tryptophan | | | |
| | | | Biosynthesis | | | |
| R1029 | anthranilate phosphori- | Ant[p] + PRPP[p] ==> PA[p] + PP[p] | Phenylalanine | 0 | 0 | 1000 |
| | bosyltransferase | | Tyrosine Tryptophan | | | |
| | | | Biosynthesis | | | |
| R1030 | phosphoribosylanthranilate | PA[p] <==> CDRP[p] | Phenylalanine | 1 | −1000 | 1000 |
| | isomerase | | Tyrosine Tryptophan | | | |
| | | | Biosynthesis | | | |
| R1031 | indole-3-glycerol- | CDRP[p] ==> CO2[p] + I3GP[p] | Phenylalanine | 0 | 0 | 1000 |
| | phosphate synthase | | Tyrosine Tryptophan | | | |
| | | | Biosynthesis | | | |
| R1032 | tryptophan synthesis | I3GP[p] + Ser[p] ==> GAP[p] + Trp[p] | Phenylalanine | 0 | 0 | 1000 |
| | | | Tyrosine Tryptophan | | | |
| | | | Biosynthesis | | | |
| R840 | UDP-glucuronate | UDPGlu[c] ==> CO2[c] + UDPXyl[c] | Starch Sucrose | 0 | 0 | 1000 |
| | decarboxylase | | Metabolism | | | |
| R842 | arabinoxylan synthesis | 2 UDPAra[c] + 3 UDPXyl[c] ==> 5 AraXyl[c] + | Starch Sucrose | 0 | 0 | 1000 |
| | (simpl.) | 5 UDP[c] | Metabolism | | | |
| R841 | UDP-arabinose 4-epimerase | UDPAra[c] <==> UDPXyl[c] | Amino Sugar Nucleotide | 1 | −1000 | 1000 |
| | | | Sugar Metabolism | | | |
| R843 | cellulose synthase (UDP- | UDPGlc[c] ==> Cel[c] + UDP[c] | Starch Sucrose | 0 | 0 | 1000 |
| | forming) (simpl.) | | Metabolism | | | |
| R838 | glucan synthase complex | UDPGlc[c] ==> Bglucan[c] + UDP[c] | Starch Sucrose | 0 | 0 | 1000 |
| | | | Metabolism | | | |
| R839 | UDP-glucose | 2 NAD[c] + UDPGlc[c] ==> 2 NADH[c] + | Starch Sucrose | 0 | 0 | 1000 |
| | 6-dehydrogenase | UDPGlu[c] | Metabolism | | | |
| R1033 | UDP-glucose | UDPGlc[c] <==> UDPGal[c] | Amino Sugar Nucleotide | 1 | −1000 | 1000 |
| | 4-epimerase | | Sugar Metabolism | | | |
| R1034 | mannose-6-phosphate | F6P[c] <==> Man6P[c] | Fructose Mannose | 1 | −1000 | 1000 |
| | isomerase | | Metabolism | | | |
| R1035 | phosphomannomutase | Man6P[c] <==> Man1P[c] | Fructose Mannose | 1 | −1000 | 1000 |
| | | | Metabolism | | | |
| R1036 | mannose-1-phosphate | GTP[c] + Man1P[c] <==> GDPMan[c] + | Fructose Mannose | 1 | −1000 | 1000 |
| | guanylyltransferase | PP[c] | Metabolism | | | |
| R1037 | nucleoside-diphosphate | ATP[c] + GDP[c] <==> ADP[c] + GTP[c] | Purine Metabolism | 1 | −1000 | 1000 |
| | kinase(cNDPkin: GDP) | | | | | |
| R1040 | pyruvate dehydrogenase | CoA[p] + NAD[p] + Pyr[p] ==> AcCoA[p] + | Glycolysis | 0 | 0 | 1000 |
| | complex | CO2[p] + NADH[p] | Gluconeogenesis | | | |
| R1041 | C140synthesis | 7 ATP[p] + 7 AcCoA[p] + 6 NADH[p] + | Lipids | 0 | 0 | 1000 |
| | | 6 NADPH[p] ==> 7 ADP[p] + C140 + 7 CoA[p] + | | | | |
| | | 6 NAD[p] + 6 NADP[p] + 7 P[p] | | | | |
| R1044 | C180synthesis | ATP[p] + AcCoA[p] + C160 + NADH[p] + | Lipids | 0 | 0 | 1000 |
| | | NADPH[p] ==> ADP[p] + C180 + CoA[p] + | | | | |
| | | NAD[p] + NADP[p] + P[p] | | | | |
| R1042 | C160synthesis | ATP[p] + AcCoA[p] + C140 + NADH[p] + | Lipids | 0 | 0 | 1000 |
| | | NADPH[p] ==> ADP[p] + C160 + CoA[p] + | | | | |
| | | NAD[p] + NADP[p] + P[p] | | | | |
| R1043 | C161synthesis | C160 + NADPH[p] ==> C161 + NADP[p] | Lipids | 0 | 0 | 1000 |
| R1045 | C181synthesis | C180 + NADPH[p] ==> C181 + NADP[p] | Lipids | 0 | 0 | 1000 |
| R1046 | C182synthesis | C181 + NADPH[p] ==> C182 + NADP[p] | Lipids | 0 | 0 | 1000 |
| R1047 | C183synthesis | C182 + NADPH[p] ==> C183 + NADP[p] | Lipids | 0 | 0 | 1000 |
| R1048 | ATP citrate lyase | ATP[c] + Cit[c] + CoA[c] ==> ADP[c] + | Lipids | 0 | 0 | 1000 |
| | | AcCoA[c] + OAA[cl + P[c] | | | | |
| R1049 | C200synthesis (cytosol) | ATP[c] + AcCoA[c] + C180 + NADH[c] + | Lipids | 0 | 0 | 1000 |
| | | NADPH[c] ==> ADP[c] + C200 + CoA[c] + | | | | |
| | | NAD[c] + NADP[c] + P[c] | | | | |
| R1003 | adenosylhomocysteinase | SAH[c] <==> ADN[c] + HOMOCys[c] | Cysteine Methionine | 1 | −1000 | 1000 |
| | | | Metabolism | | | |
| R1006 | methionine synthase | HOMOCys[c] + MTHF[c] ==> Met[c] + | Cysteine Methionine | 0 | 0 | 1000 |
| | (cMS) | THF[c] | Metabolism | | | |
| R1002 | methionine adenosyl- | ATP[c] + Met[c] ==> PP[c] + P[c] + SAM[c] | Cysteine Methionine | 0 | 0 | 1000 |
| | transferase | | Metabolism | | | |
| R1005 | homocysteine S- | HOMOCys[c] + SAM[c] ==> Met[c] + SAH[c] | Cysteine Methionine | 0 | 0 | 1000 |
| | methyltransferase | | Metabolism | | | |
| R1051 | glycerol-3-phosphate | DHAP[c] + NADH[c] <==> G3P[c] + NAD[c] | Lipids | 1 | −1000 | 1000 |
| | dehydrogenase | | | | | |
| R1053 | glycerol 3-phosphate O- | G3P[c] + acylCoA[c] ==> CoA[c] + | Lipids | 0 | 0 | 1000 |
| | acetyltransferase | acylG3P[c] | | | | |
| R1054 | 1-acylglycerol 3- | acylCoA[c] + acylG3P[c] ==> CoA[c] + | Lipids | 0 | 0 | 1000 |
| | phosphate acyltransferase | DAG3P[c] | | | | |
| R1055 | phosphatidate phosphatase | DAG3P[c] ==> DAG[c] + P[c] | Lipids | 0 | 0 | 1000 |
| R1056 | diglyceride acyltransferase | DAG[c] + acylCoA[c] ==> CoA[c] + TAG | Lipids | 0 | 0 | 1000 |
| R1052 | Long-chain-fatty-acid | ATP[c] + CoA[c] + ffa ==> AMP[c] + PP[c] + | Lipids | 0 | 0 | 1000 |
| | CoA ligase | acylCoA[c] | | | | |
| R1057 | diacylglycerol-choline | CDPChol[c] + DAG[c] ==> CMP[c] + PC[c] | Lipids | 0 | 0 | 1000 |
| | phosphotransferase | | | | | |
| R1058 | serine decarboxylase | Ser[c] ==> CO2[c] + EA[c] | Lipids | 0 | 0 | 1000 |
| R1059 | ethanolamine kinase | ATP[c] + EA[c] ==> ADP[c] + phEA[c] | Lipids | 0 | 0 | 1000 |
| R1060 | phosphoethanolamine N- | 3 SAM[c] + phEA[c] ==> 3 SAH[c] + pChol[c] | Lipids | 0 | 0 | 1000 |
| | methyltransferase | | | | | |
| R1061 | cholinephosphate | CTP[c] + pChol[c] ==> CDPChol[c] + PP[c] | Lipids | 0 | 0 | 1000 |
| | cytidylyltransferase | | | | | |
| R1062 | cytidylate kinase | ATP[c] + CMP[c] <==> ADP[c] + CDP[c] | Lipids | 1 | −1000 | 1000 |
| R1063 | nucleoside-diphosphate | ATP[c] + CDP[c] <==> ADP[c] + CTP[c] | Lipids | 1 | −1000 | 1000 |
| | kinase | | | | | |
| R1064 | phosphatidate | CTP[c] + DAG3P[c] ==> CDPDAG[c] + PP[c] | Lipids | 0 | 0 | 1000 |
| | cytidylyltransferase | | | | | |
| R1065 | CDP-diacylglycerol- | CDPDAG[c] + Ser[c] ==> CMP[c] + pSer[c] | Lipids | 0 | 0 | 1000 |
| | serine O- | | | | | |
| | phosphatidyltransferase | | | | | |
| R1066 | phosphatidylserine | pSer[c] ==> CO2[c] + PEA | Lipids | 0 | 0 | 1000 |
| | decarboxylase | | | | | |
| R1069 | phospholipid:diacylglycerol | DAG[c] + PC[c] ==> LPC + TAG | Lipids | 0 | 0 | 1000 |
| | acyltransferase | | | | | |
| R1067 | ethanolamine-phosphate | CTP[c] + phEA[c] ==> CDPEA[c] + PP[c] | Lipids | 0 | 0 | 1000 |
| | cytidylyltransferase | | | | | |
| R1068 | ethanolaminephospho- | CDPEA[c] + DAG[c] ==> CMP[c] + PEA | Lipids | 0 | 0 | 1000 |
| | transferase | | | | | |
| R1071 | glycerol-3-phosphate | DHAP[p] + NADH[p] <==> G3P[p] + NAD[p] | Lipids | 1 | −1000 | 1000 |
| | dehydrogenase (p) | | | | | |
| R1073 | glycerol 3-phosphate O- | G3P[p] + acylCoA[p] ==> CoA[p] + | Lipids | 0 | 0 | 1000 |
| | acyltransferase (p) | acylG3P[p] | | | | |
| R1074 | 1-acylglycerol 3- | acylCoA[p] + acylG3P[p] ==> CoA[p] + | Lipids | 0 | 0 | 1000 |
| | phosphate acyltransferase | DAG3P[p] | | | | |
| | (p) | | | | | |
| R1075 | phosphatidate phosphatase | DAG3P[p] ==> DAG[p] + P[p] | Lipids | 0 | 0 | 1000 |
| R1077 | UDPGalactose:DAG | DAG[p] + UDPGal[c] ==> MGDG[p] + UDP[c] | Lipids | 0 | 0 | 1000 |
| | galactosyltransferase | | | | | |
| R1078 | digalactosyldiacylglycerol | MGDG[p] + UDPGal[c] ==> DGDG[p] + | Lipids | 0 | 0 | 1000 |
| | synthase | UDP[c] | | | | |
| R1072 | Long-chain-fatty-acid | ATP[p] + CoA[p] + ffa ==> AMP[p] + PP[p] + | Lipids | 0 | 0 | 1000 |
| | CoA ligase (p) | acylCoA[p] | | | | |
| R998 | biomasssynthesis | 3.71 ATP[m] + 0.0617 Ala[c] + 0.0174 | Biomass | 0 | 0 | 1000 |
| | | AraX-yl[c] + 0.052 Arg[c] + 0.0662 Asp[c] + | | | | |
| | | 0.0076 Bglucan[c] + 0.1343 Cel[c] + 0.0177 | | | | |
| | | Cys[p] + 0.1121 Glu[c] + 0.0598 Gly[p] + | | | | |
| | | 0.015 His[p] + 0.0295 Ile[p] + 0.0624 Leu[p] + | | | | |
| | | 0.0265 Lys[p] + 0.015 Met[p] + 0.0295 Phe[p] + | | | | |
| | | 0.0407 Pro[p] + 0.0478 Ser[p] + 0.023 TAG + | | | | |
| | | 0.0313 Thr[p] + 0.0062 Trp[p] + 0.0219 Tyr[p] + | | | | |
| | | 0.0469 Val[p] + 0.0075 ffa + 4.5956 starch[p] + | | | | |
| | | 0.0477 sucrose[c] + 0.0353 pentosan + 0.0042 | | | | |
| | | PL + 0.0018 GL ==> 3.71 ADP[m] + 3.71 P[m] + | | | | |
| | | biomass | | | | |
| R1038 | PentosanProteinsynthesis | 0.0573 Ala[c] + 0.0943 Arg[c] + 0.0918 Asp[c] + | Biomass | 0 | 0 | 1000 |
| | | 0.0223 Cys[p] + 0.1718 Glu[c] + 0.0468 Gly[p] + | | | | |
| | | 0.0243 His[p] + 0.0403 Ile[p] + 0.0853 Leu[p] + | | | | |
| | | 0.0403 Lys[p] + 0.0233 Met[p] + 0.0508 Phe[p] + | | | | |
| | | 0.0488 Pro[p] + 0.0523 Ser[p] + 0.0388 Thr[p] + | | | | |
| | | 0.0133 Trp[p] + 0.0413 Tyr[p] + 0.0573 | | | | |
| | | Val[p] ==> pentosanProtein | | | | |
| R1039 | pentosanSynthesis | 0.0455 GDPMan[c] + 0.0364 UDPAra[c] + | Biomass | 0 | 0 | 1000 |
| | | 0.0455 UDPGal[c] + 0.6545 UDPGlc[c] + | | | | |
| | | 0.0909 UDPGlu[c] + 0.0364 UDPXyl[c] + | | | | |
| | | 0.091 pentosanProtein ==> 0.0455 GDP[c] + | | | | |
| | | 0.8637 UDP[c] + pentosan | | | | |
| R1050 | fattyacidsSynthesis | 0.0032 C140 + 0.1826 C160 + 0.0036 C161 + | Lipids | 0 | 0 | 1000 |
| | | 0.0212 C180 + 0.3986 C181 + 0.3679 C182 + | | | | |
| | | 0.0146 C183 + 0.0082 C200 ==> ffa | | | | |
| R1070 | phospholipidSynthesis | 0.12 LPC + 0.44 PC[c] + 0.44 PEA ==> P[c] + PL | Lipids | 0 | 0 | 1000 |
| R1079 | glycolipidSynthesis | 0.5 DGDG[p] + 0.5 MGDG[p] ==> GL | Lipids | 0 | 0 | 1000 |
| R1080 | LysineExport | Lys[p]<==> | Excretion | 1 | +1000 | 1000 |

TABLE 4
abbreviations of metabolite names

| Metabolite | | Metabolite | Metabolite |
|---|---|---|---|
| name | Metabolite description | Compartment | KEGGID |
| 13BPG[c] | 1,3-Bisphospho-D-glycerate | Cytosol | C00236 |
| 13BPG[p] | 1,3-Bisphospho-D-glycerate | Plastid | C00236 |
| 2AHB[p] | (S)-2-Aceto-2-hydroxybutanoate | Plastid | C06006 |
| 2IPM[p] | (2S)-2-Isopropylmalate | Plastid | C02504 |
| 2OB[p] | 2-Oxobutanoate | Plastid | C00109 |
| 2OG[c] | 2-Oxoglutarate | Cytosol | C00026 |
| 2OG[m] | 2-Oxoglutarate | Mitochondrion | C00026 |
| 2OG[p] | 2-Oxoglutarate | Plastid | C00026 |
| 2PG[c] | 2-Phospho-D-glycerate | Cytosol | C00631 |
| 2PG[p] | 2-Phospho-D-glycerate | Plastid | C00631 |
| 3DHQ[p] | 3-Dehydroquinate | Plastid | C00944 |
| 3DSh[p] | 3-Dehydroshikimate | Plastid | C02637 |
| 3IPM[p] | 3-Isopropylmalate | Plastid | C04411 |
| 3PG[c] | 3-Phospho-D-glycerate | Cytosol | C00197 |
| 3PG[p] | 3-Phospho-D-glycerate | Plastid | C00197 |
| 6PG[c] | 6-Phospho-D-gluconate | Cytosol | C00345 |
| 6PG[p] | 6-Phospho-D-gluconate | Plastid | C00345 |
| ADN[c] | Adenosine | Cytosol | C00212 |
| ADP[c] | ADP | Cytosol | C00008 |
| ADP[m] | ADP | Mitochondrion | C00008 |
| ADP[p] | ADP | Plastid | C00008 |
| ADPglc[c] | ADP-glucose | Cytosol | C00498 |
| ADPglc[p] | ADP-glucose | Plastid | C00498 |
| AICAR[p] | 5-Phosphoribosyl-4-carbamoyl-5-aminoimidazole | Plastid | C04677 |
| AMP[c] | AMP | Cytosol | C00020 |
| AMP[p] | AMP | Plastid | C00020 |
| ATP[c] | ATP | Cytosol | C00002 |
| ATP[m] | ATP | Mitochondrion | C00002 |
| ATP[p] | ATP | Plastid | C00002 |
| AcA[c] | Acetate | Cytosol | C00033 |
| AcA[p] | Acetate | Plastid | C00033 |
| AcAl[c] | Acetaldehyde | Cytosol | C00084 |
| AcCoA[c] | Acetyl-CoA | Cytosol | C00024 |
| AcCoA[m] | Acetyl-CoA | Mitochondrion | C00024 |
| AcCoA[p] | Acetyl-CoA | Plastid | C00024 |
| AcGluP[p] | N-Acetyl-L-glutamate 5-phosphate | Plastid | C04133 |
| AcGluSA[p] | N-Acetyl-L-glutamate 5-semialdehyde | Plastid | C01250 |
| AcGlu[p] | N-Acetyl-L-glutamate | Plastid | C00624 |
| AcLac[p] | 2-Acetolactate | Plastid | C00900 |
| AcOr[p] | N-Acetylornithine | Plastid | C00437 |
| AcSer[p] | O-Acetyl-L-serine | Plastid | C00979 |
| Agn[p] | L-Arogenate | Plastid | C00826 |
| Ala[c] | L-Alanine | Cytosol | C00041 |
| Ant[p] | Anthranilate | Plastid | C00108 |
| AraXyl[c] | Arabinoxylan | Cytosol | C01889 |
| ArgSucc[c] | L-Argininosuccinate | Cytosol | C03406 |
| Arg[c] | L-Arginine | Cytosol | C00062 |
| Asn[c] | L-Asparagine | Cytosol | C00152 |
| AspSA[p] | L-Aspartate 4-semialdehyde | Plastid | C00441 |
| Asp[c] | L-Aspartate | Cytosol | C00049 |
| Asp[m] | L-Aspartate | Mitochondrion | C00049 |
| Asp[p] | L-Aspartate | Plastid | C00049 |
| Asuc[p] | Adenylosuccinate | Plastid | C03794 |
| Bglucan[c] | beta-D-Glucan | Cytosol | C00551 |
| C140 | Myristic acid (C14:0) | | C06424 |
| C160 | Palmitic acid (C16:0) | | C00249 |
| C161 | Palmitoleic acid (C16:1) | | C08362 |
| C180 | Stearic acid (C18:0) | | C01530 |
| C181 | Oleic acid (C18:1) | | C00712 |
| C182 | Linoleic acid (C18:2) | | C01595 |
| C183 | alpha-Linolenic acid (C18:3) | | C06427 |
| C200 | Arachidonic acid (C20:0) | | C00219 |
| CDPChol[c] | CDP-choline | Cytosol | C00307 |
| CDPDAG[c] | CDP-diacylglycerol | Cytosol | C00269 |
| CDPEA[c] | CDP-ethanolamine | Cytosol | C00570 |
| CDP[c] | CDP | Cytosol | C00112 |
| CDRP[p] | 1-(2-Carboxyphenylamino)-1-deoxy-D-ribulose 5- | Plastid | C01302 |
| | phosphate | | |
| CMP[c] | CMP | Cytosol | C00055 |
| CO2[c] | CO2 | Cytosol | C00011 |
| CO2[m] | CO2 | Mitochondrion | C00011 |
| CO2[p] | CO2 | Plastid | C00011 |
| CP[p] | Carbamoyl phosphate | Plastid | C00169 |
| CTP[c] | CTP | Cytosol | C00063 |
| Cel[c] | Cellulose | Cytosol | C00760 |
| Ch[p] | Chorismate | Plastid | C00251 |
| Cit[c] | Citrate | Cytosol | C00158 |
| Cit[m] | Citrate | Mitochondrion | C00158 |
| Cit[p] | Citrate | Plastid | C00158 |
| Citru[c] | L-Citrulline | Cytosol | C00327 |
| Citru[p] | L-Citrulline | Plastid | C00327 |
| CoA[c] | CoA | Cytosol | C00010 |
| CoA[m] | CoA | Mitochondrion | C00010 |
| CoA[p] | CoA | Plastid | C00010 |
| CysTh[p] | L-Cystathionine | Plastid | C02291 |
| Cys[p] | L-Cysteine | Plastid | C00097 |
| DAG3P[c] | 1,2-Diacyl-sn-glycerol 3-phosphate | Cytosol | C00416 |
| DAG3P[p] | 1,2-Diacyl-sn-glycerol 3-phosphate | Plastid | C00416 |
| DAG[c] | 1,2-Diacyl-sn-glycerol | Cytosol | C00641 |
| DAG[p] | 1,2-Diacyl-sn-glycerol | Plastid | C00641 |
| DAH7P[p] | 2-Dehydro-3-deoxy-D-arabino-heptonate 7-phosphate | Plastid | C04691 |
| DAH[p] | LL-2,6-Diaminoheptanedioate | Plastid | C00666 |
| DGDG[p] | Digalactosyl-diacylglycerol | Plastid | C06037 |
| DHAP[c] | Glycerone phosphate | Cytosol | C00111 |
| DHAP[p] | Glycerone phosphate | Plastid | C00111 |
| DIV[p] | 2,3-Dihydroxy-isovalerate | Plastid | C04039 |
| DMV[p] | 2,3-Dihydroxy-3-methylvalerate | Plastid | C04104 |
| DPA[p] | L-2,3-Dihydrodipicolinate | Plastid | C03340 |
| E4P[p] | D-Erythrose 4-phosphate | Plastid | C00279 |
| EA[c] | Ethanolamine | Cytosol | C00189 |
| EPSP[p] | 5-O-(1-Carboxyvinyl)-3-phosphoshikimate | Plastid | C01269 |
| Eth[c] | Ethanol | Cytosol | C00469 |
| F16P[c] | D-Fructose 1,6-bisphosphate | Cytosol | C00354 |
| F16P[p] | D-Fructose 1,6-bisphosphate | Plastid | C00354 |
| F6P[c] | D-Fructose 6-phosphate | Cytosol | C00085 |
| F6P[p] | D-Fructose 6-phosphate | Plastid | C00085 |
| FTHF[c] | 10-Formyltetrahydrofolate | Cytosol | C00234 |
| FTHF[p] | 10-Formyltetrahydrofolate | Plastid | C00234 |
| For[c] | Formate | Cytosol | C00058 |
| Frc[c] | D-Fructose | Cytosol | C00095 |
| Fum[c] | Fumarate | Cytosol | C00122 |
| Fum[m] | Fumarate | Mitochondrion | C00122 |
| Fum[p] | Fumarate | Plastid | C00122 |
| G1P[c] | D-Glucose 1-phosphate | Cytosol | C00103 |
| G1P[p] | D-Glucose 1-phosphate | Plastid | C00103 |
| G3P[c] | Glycerol-3-phosphate | Cytosol | C00093 |
| G3P[p] | Glycerol-3-phosphate | Plastid | C00093 |
| G6P[c] | D-Glucose 6-phosphate | Cytosol | C00092 |
| G6P[p] | D-Glucose 6-phosphate | Plastid | C00092 |
| GAP[c] | D-Glyceraldehyde 3-phosphate | Cytosol | C00118 |
| GAP[p] | D-Glyceraldehyde 3-phosphate | Plastid | C00118 |
| GDPMan[c] | GDP-mannose | Cytosol | C00096 |
| GDP[c] | GDP | Cytosol | C00035 |
| GDP[p] | GDP | Plastid | C00035 |
| GL6P[c] | 6-Phospho-D-glucono-1,5-lactone | Cytosol | C01236 |
| GL6P[p] | 6-Phospho-D-glucono-1,5-lactone | Plastid | C01236 |
| GTP[c] | GTP | Cytosol | C00044 |
| GTP[p] | GTP | Plastid | C00044 |
| Gaba[c] | 4-Aminobutanoate | Cytosol | C00334 |
| Gaba[m] | 4-Aminobutanoate | Mitochondrion | C00334 |
| Glc[c] | D-Glucose | Cytosol | C00031 |
| Glc[p] | D-Glucose | Plastid | C00031 |
| Gln[c] | L-Glutamine | Cytosol | C00064 |
| Gln[p] | L-Glutamine | Plastid | C00064 |
| GluSA[p] | L-Glutamate 5-semialdehyde | Plastid | C01165 |
| Glu[c] | L-Glutamate | Cytosol | C00025 |
| Glu[m] | L-Glutamate | Mitochondrion | C00025 |
| Glu[p] | L-Glutamate | Plastid | C00025 |
| Glx[c] | Glyoxylate | Cytosol | C00048 |
| Gly[c] | Glycine | Cytosol | C00037 |
| Gly[m] | Glycine | Mitochondrion | C00037 |
| Gly[p] | Glycine | Plastid | C00037 |
| H2S[c] | Hydrogen sulfide | Cytosol | C00283 |
| H2S[p] | Hydrogen sulfide | Plastid | C00283 |
| HOMOCys[c] | L-Homocysteine | Cytosol | C00155 |
| HOMOCys[p] | L-Homocysteine | Plastid | C00155 |
| HOMOSer[p] | L-Homoserine | Plastid | C00263 |
| Hext | Hydron (extraplasmatic) | | C00080 |
| His[p] | L-Histidine | Plastid | C00135 |
| HolP[p] | L-Histidinol phosphate | Plastid | C01100 |
| Hol[p] | L-Histidinol | Plastid | C00860 |
| I3GP[p] | Indole-3-glycerol phosphate | Plastid | C03506 |
| IAP[p] | Imidazole-acetol phosphate | Plastid | C01267 |
| IGP[p] | D-erythro-Imidazole-glycerol phosphate | Plastid | C04666 |
| IMP[p] | Inosine monophosphate | Plastid | C00130 |
| IPO[p] | (2S)-2-Isopropyl-3-oxosuccinate | Plastid | C04236 |
| Icit[c] | Isocitrate | Cytosol | C00311 |
| Icit[m] | Isocitrate | Mitochondrion | C00311 |
| Ile[p] | L-Isoleucine | Plastid | C00407 |
| LPC | 2-Lysophosphatidylcholine | | C04230 |
| Lac[c] | L-Lactate | Cytosol | C00186 |
| Leu[p] | L-Leucine | Plastid | C00123 |
| Lys[p] | L-Lysine | Plastid | C00047 |
| METHF[c] | 5-Methyltetrahydrofolate | Cytosol | C00440 |
| METTHF[c] | 5,10-Methylenetetrahydrofolate | Cytosol | C00143 |
| METTHF[m] | 5,10-Methylenetetrahydrofolate | Mitochondrion | C00143 |
| METTHF[p] | 5,10-Methylenetetrahydrofolate | Plastid | C00143 |
| MGDG[p] | Monogalactosyl-diacylglycerol | Plastid | C03692 |
| MTHF[c] | 5-Methyltetrahydrofolate | Cytosol | C00440 |
| MTHF[p] | 5-Methyltetrahydrofolate | Plastid | C00440 |
| Mal[c] | L-Malate | Cytosol | C00149 |
| Mal[m] | L-Malate | Mitochondrion | C00149 |
| Mal[p] | L-Malate | Plastid | C00149 |
| Malt[p] | Maltose | Plastid | C00208 |
| Man1P[c] | D-Mannose 1-phosphate | Cytosol | C00636 |
| Man6P[c] | D-Mannose 6-phosphate | Cytosol | C00275 |
| Met[c] | L-Methionine | Cytosol | C00073 |
| Met[p] | L-Methionine | Plastid | C00074 |
| NAD[c] | NAD+ | Cytosol | C00003 |
| NAD[m] | NAD+ | Mitochondrion | C00003 |
| NAD[p] | NAD+ | Plastid | C00003 |
| NADH[c] | NADH | Cytosol | C00004 |
| NADH[m] | NADH | Mitochondrion | C00004 |
| NADH[p] | NADH | Plastid | C00004 |
| NADP[c] | NADP+ | Cytosol | C00006 |
| NADP[m] | NADP+ | Mitochondrion | C00006 |
| NADP[p] | NADP+ | Plastid | C00006 |
| NADPH[c] | NADPH | Cytosol | C00005 |
| NADPH[m] | NADPH | Mitochondrion | C00005 |
| NADPH[p] | NADPH | Plastid | C00005 |
| NH3[c] | NH3 | Cytosol | C00014 |
| NH3[m] | NH3 | Mitochondrion | C00014 |
| NH3[p] | NH3 | Plastid | C00014 |
| O2[c] | Oxygen | Cytosol | C00007 |
| O2[m] | Oxygen | Mitochondrion | C00007 |
| OAA[c] | Oxaloacetate | Cytosol | C00036 |
| OAA[m] | Oxaloacetate | Mitochondrion | C00036 |
| OAA[p] | Oxaloacetate | Plastid | C00036 |
| OIC[p] | 2-Oxoisocaproate | Plastid | C00233 |
| OIV[p] | 2-Oxoisovalerate | Plastid | C00141 |
| OMV[p] | 2-Oxo-3-methylvalerate | Plastid | C03465 |
| Or[p] | L-Ornithine | Plastid | C00077 |
| Oxl[c] | Oxalate | Cytosol | C00209 |
| PHOMOSer[p] | O-Phospho-L-homoserine | Plastid | C01102 |
| PA[p] | N-(5-Phospho-D-ribosyl)anthranilate | Plastid | C04302 |
| PAsp[p] | L-4-Aspartyl phosphate | Plastid | C03082 |
| PC[c] | Phosphatidylcholine | Cytosol | C00157 |
| PEA | Phosphatidylethanolamine | | C00350 |
| PEP[c] | Phosphoenolpyruvate | Cytosol | C00074 |
| PEP[p] | Phosphoenolpyruvate | Plastid | C00074 |
| PHPyr[p] | 3-Phosphohydroxypyruvate | Plastid | C03232 |
| PP[c] | Diphosphate | Cytosol | C00013 |
| PP[p] | Diphosphate | Plastid | C00013 |
| PR_AICARP[p] | Phosphoribosyl-formimino-AICAR-phosphate | Plastid | C04896 |
| PR_AMP[p] | Phosphoribosyl-AMP | Plastid | C02741 |
| PR_ATP[p] | Phosphoribosyl-ATP | Plastid | C02739 |
| PRE[p] | Prephenate | Plastid | C00254 |
| PRFICA[p] | 1-(5′-Phosphoribosyl)-5-formamido-4- | Plastid | C04734 |
| | imidazolecarboxamide | | |
| PRPP[p] | 5-Phospho-alpha-D-ribose 1-diphosphate | Plastid | C00119 |
| PRu_AICARP[p] | Phosphoribulosyl-formimino-AICAR-phosphate | Plastid | C04916 |
| P[c] | Phosphate | Cytosol | C00009 |
| P[m] | Phosphate | Mitochondrion | C00009 |
| P[p] | Phosphate | Plastid | C00009 |
| Phe[p] | L-Phenylalanine | Plastid | C00079 |
| Pro[p] | L-Proline | Plastid | C00148 |
| Pser[p] | 3-Phosphoserine | Plastid | C01005 |
| Pyr[c] | Pyruvate | Cytosol | C00022 |
| Pyr[m] | Pyruvate | Mitochondrion | C00022 |
| Pyr[p] | Pyruvate | Plastid | C00022 |
| PyrrC[p] | L-1-Pyrroline-5-carboxylate | Plastid | C03912 |
| QH2[m] | Ubiquinol | Mitochondrion | C00390 |
| Q[m] | Ubiquinone | Mitochondrion | C00399 |
| R5P[p] | D-Ribose 5-phosphate | Plastid | C00117 |
| Ru15P[p] | D-Ribulose 1,5-bisphosphate | Plastid | C01182 |
| Ru5P[c] | D-Ribulose 5-phosphate | Cytosol | C00199 |
| Ru5P[p] | D-Ribulose 5-phosphate | Plastid | C00199 |
| S6P[c] | Sucrose 6′-phosphate | Cytosol | C02591 |
| S7P[p] | Sedoheptulose 7-phosphate | Plastid | C05382 |
| SAH[c] | S-Adenosyl-L-homocysteine | Cytosol | C00021 |
| SAM[c] | S-Adenosyl-L-methionine | Cytosol | C00019 |
| Ser[c] | L-Serine | Cytosol | C00065 |
| Ser[m] | L-Serine | Mitochondrion | C00065 |
| Ser[p] | L-Serine | Plastid | C00065 |
| Sh3P[p] | Shikimate 3-phosphate | Plastid | C03175 |
| Sh[p] | Shikimate | Plastid | C00493 |
| SuccAH[p] | N-Succinyl-2-L-amino-6-oxoheptanedioate | Plastid | C04462 |
| SuccCoA[m] | Succinyl-CoA | Mitochondrion | C00091 |
| SuccCoA[p] | Succinyl-CoA | Plastid | C00091 |
| SuccDAH[p] | N-Succinyl-LL-2,6-diaminoheptanedioate | Plastid | C04421 |
| SuccSAl[m] | Succinate semialdehyde | Mitochondrion | C00232 |
| Succ[c] | Succinate | Cytosol | C00042 |
| Succ[m] | Succinate | Mitochondrion | C00042 |
| Succ[p] | Succinate | Plastid | C00042 |
| TAG | Triacylglycerol | | C00422 |
| THDPA[p] | 2,3,4,5-Tetrahydrodipicolinate | Plastid | C03972 |
| THF[c] | Tetrahydrofolate | Cytosol | C00101 |
| THF[m] | Tetrahydrofolate | Mitochondrion | C00101 |
| THF[p] | Tetrahydrofolate | Plastid | C00101 |
| Thr[p] | L-Threonine | Plastid | C00188 |
| Trp[p] | L-Tryptophan | Plastid | C00078 |
| Tyr[p] | L-Tyrosine | Plastid | C00082 |
| UDPAra[c] | UDP-L-arabinose | Cytosol | C00935 |
| UDPGal[c] | UDP-D-galactose | Cytosol | C00052 |
| UDPGlc[c] | UDP-glucose | Cytosol | C00029 |
| UDPGlu[c] | UDP-glucuronate | Cytosol | C00167 |
| UDPXyl[c] | UDP-D-xylose | Cytosol | C00190 |
| UDP[c] | UDP | Cytosol | C00015 |
| UTP[c] | UTP | Cytosol | C00075 |
| Val[p] | L-Valine | Plastid | C00183 |
| X5P[c] | D-Xylose-5-phosphate | Cytosol | C06814 |
| X5P[p] | D-Xylose-5-phosphate | Plastid | C06814 |
| acylCoA[c] | Acyl-CoA | Cytosol | C00040 |
| acylCoA[p] | Acyl-CoA | Plastid | C00040 |
| acylG3P[c] | 1-Acyl-sn-glycerol 3-phosphate | Cytosol | C00681 |
| acylG3P[p] | 1-Acyl-sn-glycerol 3-phosphate | Plastid | C00681 |
| biomass | biomass | | |
| ffa | free fatty acids | | |
| mDAH[p] | meso-2,6-Diaminoheptanedioate | Plastid | C00680 |
| pChol[c] | Phosphorylcholine | Cytosol | C00588 |
| pSer[c] | Phosphatidylserine | Cytosol | C02737 |
| phEA[c] | Phosphoethanolamine | Cytosol | C00346 |
| starch[p] | Starch | Plastid | C00369 |
| sucrose[c] | Sucrose | Cytosol | C00089 |
| pentosan | pentosan | | |
| pentosanProtein | pentosan protein | | |
| PL | phospholipids | | |
| GL | glycolipids | | |

1. A method for identifying at least one metabolic conversion step, the modulation of which increases the amount of a metabolite of interest in a plant cell, plant or plant part, said method comprising:
(a) establishing a stoichiometric network model for the metabolism of the plant cell, plant or plant part including the synthesis pathway for the metabolite of interest;
(b) identifying at least one candidate metabolic conversion step by applying at least one algorithm of Growth-coupled Design; and
(c) validating the at least one candidate metabolic conversion step by a constraint-based modeling approach in the stoichiometric network model, wherein an increase in the metabolite of interest occurring in said constraint-based modeling approach is indicative for a metabolic conversion step, the modulation of which increases the amount of the metabolite of interest in the plant cell, plant or plant part.

2. The method of claim 1, wherein said modulation of a metabolic conversion step encompasses decreasing or increasing the activity of at least one enzyme catalyzing the metabolic conversion step in the plant cell.

3. The method of claim 1, wherein said stoichiometric network model for the metabolism of the plant cell, plant or plant part comprises all relevant metabolic conversion steps of the anabolic and catabolic pathways of the metabolism of the plant cell, plant or plant part and wherein each metabolic conversion step is defined by its underlying reaction stoichiometry.

4. The method of claim 1, wherein said at least one algorithm for solving the Growth-coupled Design (i) is capable of at least calculating the amount of the metabolite of interest obtained in the stoichiometric network model under conditions where at least one metabolic enzymatic conversion step is reduced and (ii) is capable of thereby identifying at least one metabolic enzymatic conversion step the reduction of which yields the maximum amount for the metabolite of interest.

5. The method of claim 4, wherein the amount of the metabolite of interest is calculated based on the calculated amount of biomass.

6. The method of claim 5, wherein said amount of biomass is calculated based on (i) fixed substrate uptake rates for the metabolic network of the plant cell, plant or plant part and/or (ii) the plant-specific nutritional composition in the stoichiometric network model under conditions where at least one metabolic enzymatic conversion step is reduced or enhanced.

7. The method of claim 4, wherein said at least one algorithm for solving the Growth-coupled Design is selected from the group consisting of: OptKnock, RobustKnock and OptGene.

8. The method of claim 7, wherein OptKnock and/or RobustKnock are to be used if one to four metabolic enzymatic conversion step(s), the modulation of which increases a metabolite of interest in a plant cell, plant or plant part, shall be identified.

9. The method of claim 7, wherein OptGene is to be used if more than four metabolic enzymatic conversion steps, the modulation of which increases a metabolite of interest in a plant cell, plant or plant part, shall be identified.

10. The method of claim 1, wherein said plant cell, plant or plant part is a rice cell, rice plant, rice plant part, or rice seed.

11. The method of claim 1, wherein said metabolite of interest is an amino acid, a fatty acid, or a carbohydrate.

12. The method of claim 1, wherein steps (a) to (c) of said method are automated by implementation on a data processing device.

13. The method of claim 1, wherein said method further comprises the further step of:
(d) determining whether the metabolic enzymatic conversion step validated in step (c) increases the metabolite of interest in the plant cell, plant or plant part by modulating the said metabolic enzymatic conversion step in a plant cell, plant or plant part in vivo.

14. A method for generating a plant cell, plant or plant part which produces an increased amount of a metabolite of interest when compared to a control, said method comprising:
(a) identifying a metabolic conversion step, the modulation of which increases a metabolite of interest in a plant cell, plant or plant part, by the method of claim 1; and
(b) stably modulating the said metabolic conversion step such that the amount of the metabolite of interest is increased in vivo in a plant cell, plant or plant part.

15. A method for the manufacture of a metabolite of interest comprising the steps of the method of claim 14 and the further step of obtaining the metabolite of interest from the generated plant cell, plant or plant part.

16. A plant cell, plant or plant part obtainable by the method according to claim 14, which produces an increased amount of a metabolite of interest when compared to a control.

17. A device comprising a data processor having tangibly embedded least one of the algorithms of the invention.

18. The device of claim 17, wherein the device is a data processing device.

19. A data carrier comprising the data defining the stoichiometric network model established according to claim 1.
